(19) United States
(12) Patent Application Publication
(10) Pub. No.: US 2022/0220477 A1
(43) Pub. Date: Jul. 14, 2022

(54) METHODS OF TREATING DISEASES ASSOCIATED WITH CELLS EXHIBITING ER STRESS OR WITH NEURAL TISSUE DAMAGE

(71) Applicant: Ariel Scientific Innovations Ltd., Ariel (IL)

(72) Inventors: Ruth BIRK, Rehovot (IL); Mariana ANOSOV, Haifa (IL); Avital HORWITZ, Talmon (IL)

(73) Assignee: Ariel Scientific Innovations Ltd., Ariel (IL)

(21) Appl. No.: 17/610,172

(22) PCT Filed: May 8, 2020
(86) PCT No.: PCT/IL2020/050504

§ 371 Date: Nov. 10, 2021

Related U.S. Application Data
(60) Provisional application No. 62/969,173, filed on Feb. 3, 2020.
(60) Provisional application No. 62/845,899, filed on May 10, 2019.

(51) Int. Cl. C12N 15/113; A61P 25/28

(52) U.S. Cl. CPC C12N 15/113; C12N 2310/14; A61P 25/28

(57) ABSTRACT

Methods of treating diseases associated with cells exhibiting ER stress are provided. Accordingly, there is provided a method of treating a disease associated with cells exhibiting ER stress in a subject in need thereof, comprising administering to the subject a therapeutically effective amount of an agent which downregulates expression and/or activity of BBS. Also provided are methods of reducing a level of XBP1, spliced XBP-1, CHOP, Bip, ATF6alpha p50 and/or phosphorylated IREalpha and/or inducing cell death in a cell exhibiting ER stress. Also provided are methods of forming or regenerating a neural tissue and methods of treating a subject having a disease that can benefit from neural tissue formation or regeneration.

### RELATED APPLICATION

This application claims priority from U.S. Patent Application No. 62/845,899 filed on May 10, 2019, and U.S. Patent Application No. 62/969,173 filed on Feb. 3, 2020 the contents of which are incorporated herein by reference in their entirety.

### SEQUENCE LISTING STATEMENT

The ASCII file, entitled 82630 SEQUENCE LISTING, created on 6 May 2020, comprising 10,865 bytes, submitted concurrently with the filing of this application is incorporated herein by reference.

### FIELD AND BACKGROUND OF THE INVENTION

The present invention, in some embodiments thereof, relates to methods of treating diseases associated with cells exhibiting ER stress.

The endoplasmic reticulum (ER) is a multi-functional cellular compartment that functions in protein folding, lipid biosynthesis, and calcium homeostasis. An internal or external cellular insult that compromises ER homeostasis by stressing its protein folding capacity, resulting in accumulation of misfolded and unfolded proteins, is termed “ER stress.” Cells cope with ER stress by activating an ER stress signaling network called the Unfolded Protein Response (UPR). Basically, the UPR initiates by Grp78 recruitment to chaperone the misfolded proteins, resulting in Grp78 dissociation from its conformational binding state of the transmembrane receptor proteins PERK, IRE1α and ATF6α ensuing their activation. Following, the activated PERK phosphorylates eIF2α, to thereby inhibit translation; the activated phosphorylated IRE1α cleaves the 26 bp intron from XBP1, facilitating its translation; and the activated ATF6α translocates to the Golgi, where it is cleaved by proteases to form an active 50 kDa fragment (ATF6α p50). Following, ATF6α p50 and XBP1 bind ERSE promoters in the nucleus to produce upregulation of the proteins involved in the UPR. The UPR has three aims: initially to restore normal function of the cell by halting protein translation, degrading misfolded proteins, and activating the signaling pathways that lead to increasing the production of molecular chaperones involved in protein folding. If these objectives are not achieved within a certain time span or the disruption is prolonged, the UPR aims towards cell death.

Evidence suggests that chronic ER stress is of major importance in the pathogenesis of numerous conditions, such as cancer, infections, inflammatory diseases, neurodegeneration and metabolic diseases such as diabetes mellitus and obesity. Hence, targeting components of the UPR, such as XBP-1 and IRE1α, has been suggested for treatment of such diseases [see e.g. Cubillos-Ruiz et al., (2017) Cell. 168(4): 692-706].

Bardet-Biedl syndrome (BBS; OMIM 209900) is a clinically and genetically heterogeneous, autosomal recessive, ciliopathy disorder⁽¹⁾. The primary BBS characteristics include: obesity, polydactyly, rod-cone dystrophy, genital abnormalities, renal defects and learning difficulties⁽³⁾. Loss or dysfunction of any of 21 BBS proteins (BBS1-21) identified to date has been shown to cause the full multi-systemic features of the syndrome⁽²⁾. The major identified role of the BBS proteins is in the primary cilium-centrosome complex involved in the formation and function of primary cilium⁽⁶⁾. Eight out of the 21 BBS proteins (1, 2, 4, 5, 7, 8, 9, and 18) form a complex called ‘BBSome’⁽⁷⁾, which plays an important role in transporting ciliary components to the base of the cilium and vesicle trafficking. An additional BBS protein complex which functions as a BBSome chaperone includes; BBS6, BBS10 and BBS12⁽⁸⁾. However, BBS proteins have been associated with other and varied extraciliary functions. For example; BBS3 is part of ADP-ribosylation factor-like proteins family (ARLs), which are involved in protein trafficking⁽⁹⁾; BBS7 interacts with the polycomb group (PcG) member RNF2 (10); BBS11 is an E3 ubiquitin ligase TRIM32 involved in protein ubiquitination (11); BBS12 depletion in retinal explants leads to photoreceptor abnormalities (12); BBS14 (CEP290) associates with microtubule-based transport proteins (13); and BBS1 interacts with leptin receptor trafficking (14).

Additional background art includes:
Forti, E. et al. (2007) The international journal of biochemistry & cell biology, 39(5), 1055-1062;
Nahum, N. et al. (2017) IUBMB life, 69(7), 489-499;
Aksanov, O. et al. (2014) Cellular and molecular life sciences, 71(17), 3381-3392;
Marion, V. et al. (2009) Proceedings of the National Academy of Sciences, 106(6), 1820-1825;
US Application Publication No: US20170231930;
US Application Publication No: US20170151196;
US Application Publication No: US20030170645;
US Application Publication No: US20060110761;
US Application Publication No: US20100130429; and
US Application Publication No: US20060134649.

### SUMMARY OF THE INVENTION

According to an aspect of some embodiments of the present invention there is provided a method of treating a disease associated with cells exhibiting ER stress in a subject in need thereof, the method comprising administering to the subject a therapeutically effective amount of an agent which downregulates expression and/or activity of BBS, thereby treating the disease associated with cells exhibiting ER stress in the subject.

According to an aspect of some embodiments of the present invention there is provided an agent which downregulates expression and/or activity of BBS for use in the treatment of a disease associated with cells exhibiting ER stress in a subject.

According to some embodiments of the invention, the disease is selected from the group consisting of cancer, an inflammatory disease, a metabolic disease and infection.

According to an aspect of some embodiments of the present invention there is provided a method of reducing a level of XBP1, spliced XBP-1, CHOP, Bip, ATF6α p50 and/or phosphorylated IRE1α in a cell exhibiting ER stress, the method comprising contacting the cell exhibiting the ER stress with an agent which downregulates expression and/or activity of BBS, wherein the BBS is not BBS12, thereby reducing the level of XBP1, spliced XBP-1, CHOP, Bip, ATF6α p50 and/or phosphorylated IRE1α in the cell.

According to an aspect of some embodiments of the present invention there is provided a method of inducing cell death of a cell exhibiting ER stress, the method comprising contacting the cells exhibiting the ER stress with an agent which downregulates expression and/or activity of BBS, wherein the BBS is not BBS12, thereby inducing cell death of the cell.

According to an aspect of some embodiments of the present invention there is provided a method of forming or regenerating a neural tissue, the method comprising contacting neuronal stem or progenitor cells with an agent which downregulates expression and/or activity of BBS, thereby forming or regenerating the neural tissue.

According to an aspect of some embodiments of the present invention provided a method of treating a subject having a disease that can benefit from neural tissue formation or regeneration, the method comprising administering to the subject a therapeutically effective amount of an agent which downregulates expression and/or activity of BBS, thereby treating the disease that can benefit from neural tissue formation or regeneration in the subject.

According to an aspect of some embodiments of the present invention provided an agent which downregulates expression and/or activity of BBS for use in the treatment of a disease that can benefit from neural tissue formation or regeneration.

According to some embodiments of the invention, the disease is selected from the group consisting of neurodegenerative disease, ischemia, stroke, neuronal loss associated with aging and nerve injury caused by trauma.

According to some embodiments of the invention, the contacting is effected in-vitro or ex-vivo.

According to some embodiments of the invention, the contacting is effected in-vivo.

According to some embodiments of the invention, the agent is an RNA silencing agent.

According to some embodiments of the invention, the agent is an aptamer, a peptide or a small molecule.

According to an aspect of some embodiments of the present invention there is provided a method of diagnosing a disease associated with cells exhibiting ER stress in a subject, the method comprising determining a level of expression and/or activity of BBS in a biological sample of the subject, wherein a level of expression and/or activity of BBS above a predetermined threshold in the sample is indicative of the disease associated with cells exhibiting ER stress.

According to some embodiments of the invention, the BBS is not BBS12.

According to some embodiments of the invention, the BBS is selected from the group consisting of BBS1, BBS2, BBS3, BBS4, BBS5, BBS6, BBS7, BBS8, BBS9, BBS10, BBS11, BBS12, BBS13, BBS14, BBS15, BBS16, BBS17, BBS18, BBS19, BBS20 and BBS21.

According to some embodiments of the invention, the BBS is selected from the group consisting of BBS1, BBS2, BBS3, BBS4, BBS5, BBS6, BBS7, BBS8, BBS9, BBS10, BBS11, BBS13, BBS14, BBS15, BBS16, BBS17, BBS18, BBS19, BBS20 and BBS21.

According to some embodiments of the invention, the BBS is selected from the group consisting of BBS1, BBS2, BBS4, BBS5, BBS7, BBS8, BBS9 and BBS18.

According to some embodiments of the invention, the BBS comprises BBS4.

According to some embodiments of the invention, downregulating activity of the BBS comprises affecting localization of the BBS.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G demonstrate BBS4 and XBP-1 transcript and protein levels in 3T3-F442A cells treated or un-treated with Tunicamycin (TM) following three days of in-vitro differentiation. Shown are protein levels (FIGS. 1A-B and 1E-F) and mRNA levels (FIGS. 1C-D and 1G) in control cells, cells with reduced expression of BBS4 (SiBBS4) and cells over-expressing BBS4 (OEBBS4). BBS4 and XBP-1 protein levels were normalized to the housekeeping protein actin; and mRNA levels were normalized to the housekeeping gene S18/GAPDH as indicated. Results are expressed as mean f SE of 3 independent experiment (n=3). Asterisks represent significant statistical difference * P<0.05, ** P<0.01, *** P<0.001.

FIGS. 2A-E demonstrate that BBS4 protein is localized to the Endoplasmic Reticulum (ER) at day 3 of adipocytes in-vitro differentiation. FIG. 2A demonstrates no BBS4 protein in the nuclear fraction of 3T3-F442A cells treated or un-treated with TM following three days of in-vitro differentiation. HSP90 was used as a cytosolic marker and Histone H3 as a nuclear marker. Shown are western blot of 3 independent experiments (n=3). FIG. 2B demonstrates BBS4 protein levels in the ER fraction of 3T3-F442A cells treated or un-treated with TM following three days of in-vitro differentiation. Actin was used as a cytosolic marker and IRE1α as an ER marker. Shown are western blot results of 2 independent experiments (n=3). FIG. 2C demonstrates in-silico BBS4 ER localization sequences (ELS) using LocSigDB database (genome(dot)unmc(dot)edu/LocSigDB/). FIG. 2D shows confocal microscopy images of 3T3-F442A cells immunostained with an anti-BBS4 primary antibody followed by Goat Anti-Mouse IgG Alexa Fluor 488. The nucleus was visualized by DAPI staining. The images represent fields of cells (n=1000-1500); Scale bar=10 μm. FIG. 2E shows confocal microscopy images of 3T3-F442A cells immunostained with an anti-PDI primary antibody followed by Goat Anti-Mouse IgG Alexa Fluor 555. The nucleus was visualized by DAPI staining. The images represent fields of cells (n=1000-1500); Scale bar=10 μm.

FIGS. 3A-B show transmission electron micrographs of SiBBS4 3T3-F442A cells 8 days of differentiation demonstrating multiple ER swellings. M—Mitochondria, Lys—Lysosome, ER—endoplasmic reticulum. Multiple Lysosomes and ER swellings are marked by arrows. Scale bar=5000 nm in FIG. 3A and 1000 nm in FIG. 3B. Samples were examined using Jeol Jem 1230 microscope.

FIGS. 4A-B demonstrate XBP-1 transcript levels during adipocytes in-vitro differentiation. Shown is XBP-1 mRNA levels in control, SiBBS4 and OEBBS4 3T3-F442A cells at the indicated days during differentiation. Transcript levels were normalized to the housekeeping gene-S18.

FIG. 5 demonstrates the subcellular localization of XBP-1. Shown are confocal microscopy images of control and SiBBS4 3T3-F442A cells treated with TM for 6 hours and immunostained with an anti-XBP-1 primary antibody followed by Goat Anti-Mouse IgG Alexa Fluor 555. The nucleus was visualized by DAPI staining. The images represent fields of cells (n=1000-1500); Scale bar=50 μm.

FIGS. 6A-J demonstrate that XBP-1 down regulation in SiBBS4 cells occurs due to specific inhibition of ATF6α and pIRE1α. Shown are the indicated protein and transcript levels in control and SiBBS4 3T3-F442A cells treated or un-treated with TM following three days of in-vitro differentiation. FIGS. 6A-B show protein levels of full length and cleaved ATF6α normalized to the housekeeping protein actin. FIGS. 6C-D show ATF6α (FIG. 6C) and BIP (FIG. 6D) mRNA levels normalized to the housekeeping gene-S18. FIG. 6E shows SREBP1 protein levels normalized to the housekeeping protein Actin. FIGS. 6F-G show Phospho-IRE1α (P-IRE1α) protein levels normalized to the housekeeping protein Actin. FIGS. 6H-I are graphs demonstrating percentages of XBP-1 splicing. The percentage of XBP-1 splicing was calculated as the intensity of XBPs divided by the total intensities of XBP-1 un-spliced (XBPu) and XBP-1 spliced (XBPs). Results are expressed as mean f SE of 2-3 independent experiments (n=3). Asterisks represent significant statistical difference * P<0.05, ** P<0.01, *** P<0.001). FIG. 6J demonstrates the subcellular localization of ATF6a. Shown are confocal microscopy images of control and SiBBS4 3T3-F442A cells treated with TM for 2 hours and immunostained with an anti-ATF6α primary antibody followed by Goat Anti-Mouse IgG Alexa Fluor 555. The nucleus was visualized by DAPI staining. The images represent fields of cells (n=1000-1500); Scale bar=50 μm.

FIGS. 7A-H demonstrate transcript and protein levels of several apoptosis-associated genes in 3T3-F442A cells treated or untreated with TM following three days of in-vitro differentiation. Shown are CHOP protein (FIGS. 7A-D) and mRNA (FIG. 7E) levels, Caspase-3 mRNA levels (FIG. 7F), Bax mRNA levels (FIG. 7G) and Bcl-2 mRNA levels (FIG. 7H) in control SiBBS4 and OEBBS4 3T3-F442A cells, as indicated. CHOP protein levels were normalized to the housekeeping protein actin. Bax and Bcl-2 mRNA levels were normalized to the housekeeping gene S1; and caspase-3 and CHOP mRNA levels were normalized to the housekeeping gene-GAPDH. Results are expressed as mean±SE of 2 independent experiments (n=3). Asterisks represent significant statistical difference * P<0.05, ** P<0.01, *** P<0.001.

FIGS. 8A-F demonstrate ER stress UPR markers XBP-1 and CHOP during SH-SY5Y differentiation. Control and siBBS4 SH-SY5Y cells were induced to differentiate (using 10 μM RA) for 5 days (day 0—undifferentiated). Total mRNA and protein were extracted throughout the differentiation process and subjected to RT-qPCR and western blot analysis, respectively. Gene expression and protein levels were normalized to the housekeeping gene GAPDH and housekeeping protein actin, respectively. FIG. 8A demonstrates XBP-1 mRNA levels. FIG. 8B demonstrates XBP-1 protein levels. FIG. 8C demonstrates CHOP mRNA levels. FIG. 8D demonstrates CHOP protein levels. FIG. 8E demonstrates levels of spliced XBP-1 (sXBP-1) and un-spliced XBP-1 (uXBP-1). FIG. 8F demonstrates quantification of the percentages of sXBP-1. The percentage of XBP-1 splicing was calculated as the intensity of sXBP-1 divided by total intensities of XBP-1 (sXBP-1 and uXBP-1). Results are expressed as mean f SE of 3 independent experiments (n=3). Asterisks represent significant statistical difference * P<0.05 between cell models, ** P<0.05 compared to day 0.

FIGS. 9A-D demonstrate the effect of BBS4 on ATF6 localization in SH-SY5Y cells following TM-induced ER stress. FIG. 9A shows representative images demonstrating subcellular localization of ATF6. Immunohistochemistry staining was performed on siBBS4 and control cells and visualized by immunofluorescence labeling and confocal microscopy by anti-ATF6 primary antibody followed by Goat Anti-Mouse IgG Alexa Fluor 555 and by DAPI nucleus staining. Images represent fields of cells (n=1000-1500). FIG. 9B shows quantification of ATF6 nuclear localization using imageJ software. For each treatment (control, control+TM, siBBS4, siBBS4+TM), 200-250 cells were randomly chosen for nuclear intensity analysis of ATF6 in the nucleus compartment only. FIGS. 9C-D demonstrate mRNA and protein levels in siBBS4 and control SH-SY5Y cells in the presence (+TM) or absence (−TM) of TM-induced ER stress. Total mRNA and protein were extracted and subjected to RT-qPCR and western blot analysis, respectively. ATF6 expression and protein levels were normalized to the housekeeping gene GAPDH and the housekeeping protein actin. FIG. 9C demonstrates ATF6 transcript levels. FIG. 9D demonstrates full length and cleaved ATF6 protein levels. Results are expressed as mean f SE of 3 independent experiments (n=3). Asterisks represent significant statistical difference * P<0.05, ** P<0.01, *** P<0.001.

FIGS. 10A-I demonstrate the effect of BBS4 on ER stress markers in SH-SY5Y cells following TM-induced ER stress. FIGS. 10A-G demonstrate mRNA and protein levels in siBBS4 and control SH-SY5Y cells in the presence (+TM) or absence (−TM) of TM-induced ER stress. Total mRNA and protein were extracted and subjected to RT-qPCR and western blot analysis, respectively. Gene expression and protein levels were normalized to the housekeeping gene GAPDH and the housekeeping protein HSP90 or actin, respectively. FIG. 10A demonstrates BIP mRNA expression levels. FIG. 10B demonstrates CHOP mRNA expression levels. FIG. 10C demonstrates CHOP protein levels. FIG. 10D demonstrates XBP-1 mRNA expression levels. FIG. 10E demonstrates XBP-1 protein levels. FIG. 10F demonstrates percentages of sXBP-1 levels. The percentage of XBP-1 splicing was calculated as the intensity of sXBP-1 divided by total intensities of XBP-1 (sXBP-1 and uXBP-1). FIG. 10G demonstrates p-IRE-1 protein levels. Results are expressed as mean f SE of 3 independent experiments (n=3). FIG. 10H shows representative images demonstrating the subcellular localization of XBP-1. Immunohistochemistry staining was performed on siBBS4 and control cells and visualized by immunofluorescence labeling and confocal microscopy by anti-XBP-1; and by DAPI nucleus staining. Images represent fields of cells (n=1000-1500). FIG. 10I demonstrates quantification of XBP-1 nuclear localization using imageJ software. For each treatment (control, control+TM, siBBS4, siBBS4+TM) 200-250 cells were randomly chosen for nuclear intensity analysis of XBP-1 in the nucleus compartment only. Asterisks represent significant statistical difference * P<0.05 ** P<0.01 *** P<0.001.

FIGS. 11A-E demonstrate the effect of BBS4 on apoptosis markers in SH-SY5Y cells following TM-induced ER stress. Shown mRNA levels in siBBS4 and control SH-SY5Y cells in the presence (+TM) or absence (−TM) of TM-induced ER stress. Total mRNA was extracted and subjected to RT-qPCR analysis. Gene expression levels were normalized to the housekeeping gene GAPDH. FIG. 11A demonstrates Bcl-2 levels. FIG. 11B demonstrates Bax levels. FIG. 11C demonstrates Bax/Bcl-2 ratio. FIG. 11D demonstrates Caspase-3 levels. FIG. 11E demonstrates % viability 24 hours following TM treatment. Viable and dead cells were distinguished by trypan blue exclusion test. Results are expressed as mean±SE of 3 independent experiments (n=3). Asterisks represent statistically significant difference * P<0.05, ** P<0.01, *** P<0.001.

FIGS. 12A-C demonstrate BBS4 levels during neural differentiation. Control and siBBS4 SH-SY5Y or PC-12 cells were seed on 6 wells plates and induced to differentiation using 10 μM RA for 5 days (SH-SY5Y) or 50 ng/ml NGF for 8 days (PC-12). Total RNA was extracted during differentiation and subjected to RT-qPCR. BBS4 mRNA expression levels were normalized to the housekeeping gene GAPDH. Total protein was extracted during differentiation and subjected to western blot analysis. BBS4 protein levels were normalized to the housekeeping protein actin or HSP90. FIG. 12A demonstrates BBS4 mRNA levels in SH-SY5Y cells. FIG. 12B demonstrates BBS4 protein levels in SH-SY5Y cells. FIG. 12C demonstrates BBS4 protein levels in PC-12 cells. Results are expressed as mean f SE of 3 independent experiments (n=3). Asterisks represent significant statistical difference * P<0.05 between cell models, ** represent significant statistical difference P<0.05 between day 0-5.

FIGS. 13A-B demonstrate the effect of BBS4 on proliferation of SH-SY5Y (FIG. 13A) and PC-12 (FIG. 13B) neuronal cells. Equal numbers of cells were cultured and differentiated with neuronal differentiation medium. Cells number was evaluated at the indicated time points throughout differentiation using hemocytometer. Results are expressed as mean cell number pre ml f SE of 3 independent experiments (n=3). Asterisks represent significant statistical difference between cell models * P<0.05.

FIG. 14 demonstrates the effect of BBS4 on migration rate of SH-SY5Y cells, as determined by a wound-healing assay. Shown is the migration rate measured in pixels per hour by ImageJ software. Results are expressed as mean f SD of 3 independent experiments (n=3). Asterisks represent significant statistical difference between cell models *** P<0.001. Briefly, identical numbers of siBBS4 SH-SY5Y cells or control SH-SY5Y cells were seeded in 12 wells plates and incubated in NUAIR us Auto flow CO2 Water-Jacketed Incubator to 100% confluence and thereafter were subjected to wound-healing assay. A linear wound “scratch” was created using a 200 μl pipette tip. Cells migration was analyzed by continues photography starting immediately following a linear wound “scratch” was created and every 15 minutes for 10 hours using the Olympus IX81 microscope (×10). The gap area was measured in pixels and the migration rate was measured in pixels per hour by ImageJ software.

FIG. 15 demonstrates the effect of BBS4 on morphological appearance of SH-SY5Y cells, during differentiation. Undifferentiated control and siBBS4 SH-SY5Y cells were differentiated with 10 μl retinoic acid and photographed at day 0-8 of differentiation, as indicated. All images were taken using an Olympus microscope at ×20 magnification.

FIG. 16 demonstrates the effect of BBS4 on morphological appearance of PC-12 cells, during differentiation. Undifferentiated control and siBBS4 PC-12 cells were differentiated using 50 ng/μl NGF and photographed at days 0-9 of differentiation. All images were taken using an Olympus microscope at ×20 magnification.

FIGS. 17A-B demonstrates the effect of BBS4 on morphological appearance of SH-SY5Y (FIG. 17A) and PC-12 (FIG. 17B) cells, during differentiation, as assessed by neurite outgrowth. Control and siBBS4 SH-SY5Y cells were differentiated with 10 μM RA for 8 days. Control and siBBS4 PC-12 cells were differentiated with 50 ng/μl NGF for 11 days. Cells were photographed during differentiation using an Olympus microscope at ×20 magnitude, and neurite length was measured using imageJ software. Cells bearing at least one neurite with length equivalent to the cell bodies were considered as differentiated cells. More than 400 cells from at least four different fields were analyzed for each model. Results are expressed as mean±SE of 2 independent experiments (n=2). Asterisks represent significant statistical difference between cell models * P<0.05, ** P<0.01, *** P<0.001.

FIGS. 18A-C demonstrate effect of BBS4 on Nestin transcript levels during neural differentiation. Control and siBBS4 SH-SY5Y or PC-12 cells were seed on 6 wells plates and induced to differentiate using 10 μM for 5 days (SH-SY5Y) or 50 ng/ml NGF for 8 days (PC-12). Total RNA was extracted and subjected to RT-qPCR. Nestin mRNA levels were normalized to the housekeeping gene GAPDH. Total protein was extracted during differentiation and subjected to western blot analysis. Nestin protein levels were normalized to the housekeeping protein actin. FIG. 18A demonstrates Nestin mRNA levels in SH-SY5Y cells. FIG. 18B demonstrates Nestin protein levels in SH-SY5Y cells. FIG. 18C demonstrates Nestin protein levels in PC-12 cells. Results are expressed as mean±SE of 3 independent experiments (n=3). Asterisks represent significant statistical difference * P<0.05 between cell models, ** represent significant statistical difference P<0.05 between day 0-5/8.

### DESCRIPTION OF SPECIFIC EMBODIMENTS OF THE INVENTION

The present invention, in some embodiments thereof, relates methods of treating diseases associated with cells exhibiting ER stress.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. ER stress is a condition caused by internal or external cellular insult that compromises ER homeostasis and characterized by accumulation of misfolded and unfolded proteins. Cells cope with ER stress by activating a signaling network called the Unfolded Protein Response (UPR). Evidence suggests that chronic ER stress is of major importance in the pathogenesis of numerous conditions, such as cancer, infections, inflammatory diseases, neurodegeneration and metabolic diseases.

Bardet-Biedl syndrome (BBS; OMIM 209900) is a clinically and genetically heterogeneous, autosomal recessive, ciliopathy disorder⁽¹⁾. Loss or dysfunction of any of 21 BBS proteins (BBS1-21) identified to date has been shown to cause the full multi-systemic features of the syndrome⁽²⁾. The major identified role of the BBS proteins is in the primary cilium-centrosome complex involved in the formation and function of primary cilium⁽⁶⁾. However, BBS proteins have been associated with other and varied extraciliary functions.

Whilst reducing specific embodiments of the present invention to practice, the present inventors have now uncovered that expression and localization of the BBS proteins (e.g. BBS4) are responsive to ER stress and that their downregulation affects ER stress UPR in both adipocytes and neuronal cells. Furthermore, downregulation of BBS proteins (e.g. BBS4) results in increased differentiation, proliferation and migration of neuronal cells.

As is illustrated hereinunder and in the examples section, which follows, the present inventors show (Examples 1-2 of the Examples section which follows) that BBS4 protein and transcript levels are significantly up-regulated in differentiating adipocytes following Tunicamycin (TM)-induced ER stress, indicating responsiveness of BBS4 to ER stress. Furthermore, BBS4 is localized to the ER at day 3 of adipogenesis and participates in UPR activation through ATF6α and IRE1α regulation. Further, BBS4 depletion in adipocytes results in morphological changes, depletion of cleaved ATF6α and consequently of IRE1α phosphorylation and XBP-1 reduction. Following, the present inventors used a neural differentiation assay and show (Example 3 of the Examples section which follows) that in undifferentiated state, BBS4 silencing results in significantly reduced expression of ER stress markers (namely CHOP, XBP-1, cleaved ATF6, spliced XBP-1, BIP, pIRE1α) and reduced translocation of sXBP-1 and the activated cleaved ATF6 to the nucleus, under both non-stressed and TM-induced ER stress states. Furthermore, BBS4 silencing and ER stress induction results in significant upregulation of transcript levels of apoptosis markers (Bax, Bcl-2, Caspase-3), corresponding to decreased viability.

Consequently, specific embodiments of the present teachings suggest downregulating expression and/or activity of a BBS protein (e.g. BBS4) for treating a disease associated with cells exhibiting ER stress.

In addition, as is illustrated hereinunder and in the examples section, which follows, the present inventors show that BBS4 protein and transcript levels are down-regulated during neural differentiation; and that BBS4 silencing increases differentiation, proliferation and migration of neuronal cells (Example 4 of the Examples section which follows).

Consequently, specific embodiments of the present teachings suggest downregulating expression and/or activity of a BBS protein (e.g. BBS4) for forming or regenerating a neural tissue or for treating a disease that can benefit from same. Thus, according to an aspect of the present invention there is provided a method of treating a disease associated with cells exhibiting ER stress in a subject in need thereof, the method comprising administering to the subject a therapeutically effective amount of an agent which downregulates expression and/or activity of BBS, thereby treating the disease associated with cells exhibiting ER stress in the subject.

According to an alternative or an additional aspect of the present invention, there is provided an agent which downregulates expression and/or activity of BBS for use in the treatment of a disease associated with cells exhibiting ER stress in a subject.

The term “treating” or “treatment” refers to inhibiting, preventing or arresting the development of a pathology (disease, disorder or condition e.g. a disease associated with cells exhibiting ER stress) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age and of any gender which suffer from the pathology. According to specific embodiments, this term encompasses individuals who are at risk to develop the pathology.

According to specific embodiments, the subject is a human subject.

As used herein the phrase “disease associated with cells exhibiting ER stress” means that cells exhibiting ER stress drive onset and/or progression of the disease.

The term “ER stress” refers to an imbalance between the demand that a load of proteins makes on the ER and the actual folding capacity of the ER to meet that demand, manifested by accumulation of misfolded and unfolded proteins in the ER lumen.

To alleviate ER stress, cells activate a signaling network called the Unfolded Protein Response (UPR). Thus, according to specific embodiments, the cells exhibiting the ER stress have an active unfolded protein response (UPR). The “Unfolded Protein Response (UPR)” is an adaptive response to ER stress manifested by halting protein translation, degrading misfolded proteins and activating signaling pathways that lead to increased production of molecular chaperones and catalysts involved in protein folding. The UPR also regulates both survival and death factors that govern whether the cell will live or not depending on the severity of the ER stress condition.

Methods for determining ER stress and/or activation of a UPR are known in the art and disclosed for examples in Oslowski et al. Methods Enzymol. (2011; 490: 71-92, the contents of which are fully incorporated herein by reference; and include for examples, determining expression of ER stress response genes e.g. XBP1, CHOP, GRP78 (BIP), phosphorylated IRE1α, ATF6α; measuring XBP1 splicing; determining expression of apoptotic or pro-apoptotic genes e.g. Bax, Bcl-2, Caspase-3; detecting ER dilation by electron microscopy; and/or Real-time redox measurements.

As shown in the Examples section, which follows, downregulation of BBS4 reduced expression of the ER stress markers XBP-1, spliced XBP-1, CHOP, Bip, cleaved ATF6 and pIRE1α).

Hence, according to specific embodiments, the disease associated with cells exhibiting ER stress can benefit from reducing a level of XBP1, spliced XBP-1, CHOP, Bip, ATF6α p50 and/or phosphorylated IRE1α.

According to specific embodiments, the disease associated with cells exhibiting ER stress can benefit from reducing a level of XBP1, ATF6α p50 and/or phosphorylated IRE1α.

Alternatively or additionally, according to an aspect of the present invention, there is provided a method of reducing a level of XBP1, spliced XBP-1, CHOP, Bip, ATF6α p50 and/or phosphorylated IRE1α in a cell exhibiting ER stress, the method comprising contacting the cell exhibiting the ER stress with an agent which downregulates expression and/or activity of BBS, wherein said BBS is not BBS12, thereby reducing the level of XBP1, spliced XBP-1, CHOP, Bip, ATF6α p50 and/or phosphorylated IRE1α in the cell.

The contacting of some embodiments of the invention may be effected in-vitro, ex-vivo and/or in-vivo.

According to specific embodiments, the contacting is effected in-vitro or ex-vivo.

According to other specific embodiments, the contacting is effected in-vivo.

According to specific embodiments, the cell is a human cell.

As used herein the phrase “reducing a level of XBP1, spliced XBP-1, CHOP, Bip, ATF6α p50 and/or phosphorylated IRE1α” refers to a decrease in the expression level of the respective polypeptides in the presence of the agent in comparison to same in the absence of the agent. Methods of determining expression levels are known in the art and include but are not limited to PCR, ELISA or Western blot analysis.

“XBP1”, also known as X-box binding protein 1 and Tax-Responsive Element-Binding Protein 5, is a transcription factor encoded by the XBP1 gene (Gene ID 7494).

“spliced XBP-1” refers to the spliced processed form of XBP-1 resulting from excision of an intron from XBP-1 mRNA by the ER transmembrane endoribonuclease and IRE1α. In murine and human cell for example, a 26 nucleotides long intron is excised. According to specific embodiments, the spliced XBP-1 refers to the human spliced-XBP-1 such as provided in the following GeneBank Number NP_001073007.

“CHOP”, also known as C/EBP homologous protein (CHOP)” or “DNA damage-inducible transcript 3” is a pro-apoptotic transcription factor that is encoded by the DDIT3 gene (Gene ID 1649).

“Bip”, also known as “Binding immunoglobulin protein”, “heat shock 70 kDa protein 5 (HSPA5)” or “GRP-78” is a molecular chaperone encoded by the HSPA5 gene (Gene ID 3309).

“ATF6α”, also known as “activating transcription factors 6a”, is a transcription factor encoded by the ATF6 gene (Gene ID 22926).

“ATF6 p50” is the active form of ATF6α formed by proteolytic cleavage of ATF6α N-terminal cytoplasmic domain by the S2P serine protease in response to ER stress.

“IRE1α”, also known as Inositol-Requiring Enzyme 1, Endoplasmic Reticulum To Nucleus Signaling 1 and Serine/Threonine-Protein Kinase/Endoribonuclease IRE1, classified as EC 2.7.11, is an enzyme possessing both kinase and RNAse activity required for specific splicing of XBP1 mRNA, encoded by the ERN1 gene (Gene ID 2081).

“phosphorylated IRE1α” is the phosphorylated form of IRE1α which is considered to have increased RNase splicing activity.

As shown in the Examples section, which follows, downregulation of BBS4 in combination with ER stress induction upregulated transcript levels of apoptosis markers (Bax, Bcl-2, Caspase-3), corresponding to decreased viability.

Hence, according to specific embodiments, the disease associated with cells exhibiting ER stress can benefit from inducing cell death of cells associated with the disease.

Alternatively or additionally, according to an aspect of the present invention, there is provided a method of inducing cell death of a cell exhibiting ER stress, the method comprising contacting the cells exhibiting the ER stress with an agent which downregulates expression and/or activity of BBS, wherein said BBS is not BBS12, thereby inducing cell death of the cell.

As used herein the phrase “inducing cell death” refers to an increase in cell death in the presence of the agent in comparison to same in the absence of the agent. Methods of monitoring cell death are well known in the art and include, but not limited to light and electron microscopy, flow cytometry, DNA laddering, lactate dehydrogenase enzyme release, MTT/XTT enzyme activity, TUNEL assay [Roche, Mannheim, Germany]; the Annexin V assay [ApoAlert® Annexin V Apoptosis Kit (Clontech Laboratories, Inc., CA, USA)]; as well as various RNA and protein detection methods which detect level of expression and/or activity of cell death markers (e.g. Bax, Bcl-2, CHOP, caspase-3).

According to specific embodiments, cell death comprises apoptotic cell death.

According to specific embodiments, cell death comprises necrotic cell death.

Non-limiting examples of diseases associated with cells exhibiting ER stress include but are not limited to cancer, an inflammatory disease, a metabolic disease (e.g. diabetes, the metabolic syndrome, obesity), infection, neurodegenerative disorder (e.g. Alzheimer's disease, Parkinson's disease, Huntington, amyotrophic lateral sclerosis, prion disease), Wolcott-Rallison syndrome, Wolfram Syndrome, ischemia/reperfusion injury, stroke, atherosclerosis, hypoxia and hypoglycemia.

According to specific embodiments, the disease is selected from the group consisting of cancer, an inflammatory disease, a metabolic disease, infection, neurodegenerative disorder and an injury.

According to specific embodiments, the disease is selected from the group consisting of cancer, an inflammatory disease, a metabolic disease and infection.

According to specific embodiments the disease is an inflammatory disease.

Inflammatory diseases—Include, but are not limited to, chronic inflammatory diseases and acute inflammatory diseases.

Inflammatory diseases associated with hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March, 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. el al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and Pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Komberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:5107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Non-limiting examples of cancers can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

According to specific embodiments, the disease associated with cells exhibiting ER stress is a protein folding/misfolding disease such as, but not limited to, Alzheimer's disease, Parkinson's disease, amyotrophic lateral sclerosis (ALS), Creutzfeldt-Jakob disease, bovine spongiform encephalopathy (BSE), light chain amyloidosis (AL), Huntington's disease, spinobulbar muscular atrophy (Kennedy disease), Machado-Joseph disease, dentatorubral-pallidoluysian atrophy (Haw River Syndrome), spinocerebellar ataxia and the like.

As the present inventors discovered that silencing BBS4 induces proliferation, differentiation and migration of neural progenitor cells, specific embodiments suggest the disease is a disease that can benefit from neural tissue formation or regeneration.

Thus, according to an aspect of the present invention, there is provided a method of treating a subject having a disease that can benefit from neural tissue formation or regeneration, the method comprising administering to the subject a therapeutically effective amount of an agent which downregulates expression and/or activity of BBS, thereby treating the disease that can benefit from neural tissue formation or regeneration in the subject.

Alternatively or additionally, according to an aspect of the present invention, there is provided an agent which downregulates expression and/or activity of BBS for use in the treatment of a disease that can benefit from neural tissue formation or regeneration.

Alternatively or additionally, according to an aspect of the present invention, there is provided a method of forming or regenerating a neural tissue, the method comprising contacting neuronal stem or progenitor cells with an agent which downregulates expression and/or activity of BBS, thereby forming or regenerating the neural tissue.

The phrase “neuronal stem or progenitor cells”, refers to cells capable of undergoing mitotic division and differentiating into fully differentiated neurons or remaining in an undifferentiated state.

Neuronal stem or progenitor cells can be isolated using various methods known in the arts such as those disclosed by Svendsen et al. (1999) Brain Pathol. 9(3): 499-513. Rietze and Reynolds (2006) Methods Enzymol. 419:3-23; and “Handbook of Stem Cells” edit by Robert Lanze, Elsevier Academic Press, 2004.

According to specific embodiments, the neuronal stem or progenitor cells are human neuronal stem or progenitor cells.

As used herein the phrase “disease that can benefit from neural tissue formation or regeneration” refers to any disorder, disease or condition exhibiting neural tissue damage (i.e., non-functioning tissue, broken tissue, fractured tissue, fibrotic tissue, or ischemic tissue) or neural tissue loss (e.g., following a trauma, an infectious disease, a genetic disease, and the like) which require tissue generation of regeneration. Examples of diseases requiring tissue regeneration include, but are not limited to, neurodegenerative disease (e.g. Alzheimer's disease, frontotemporal dementia, dementia with Lewy bodies, corticobasal degeneration, progressive supranuclear palsy, prion disorders such as Creutzfeldt-Jakob disease, Parkinson's disease, Huntington's disease, multiple system atrophy, amyotrophic lateral sclerosis, hereditary spastic paraparesis, spinocerebellar atrophies, Friedreich's ataxia, multiple sclerosis, Charcot Marie Tooth, ALS/PCD of Guam, Down syndrome, myotonic dystrophy, Pick's disease, postencephalitic parkinsonism, primary progressive ataxia, subacute sclerosis panencephalitis, FTD-17, argyrophilic grain disease, type C Niemann-Pick's disease, Hallervorden-Spatz disease, subacute sclerosing panencephalitis, Fukuyama congenital muscular dystrophy, Kufs's disease, Cockayne syndrome, Williams syndrome, mental depression and inclusion body myositis), ischemia, stroke, neuronal loss associated with aging and nerve injury caused by trauma (e.g. spinal cord injury, traumatic brain injury and traumatic optic neuropathy). According to specific embodiments, the disease is selected from the group consisting of neurodegenerative disease, ischemia, stroke, neuronal loss associated with aging and nerve injury caused by trauma.

According to specific embodiments, the disease is selected from the group consisting of ischemia, stroke, neuronal loss associated with aging and nerve injury caused by trauma.

According to specific embodiments, the disease is not a neurodegenerative disease.

According to specific embodiments, the disease is not a retinal degeneration disease.

According to specific embodiments, the disease it not obesity.

According to specific embodiments, the cells exhibiting ER stress are not adipocytes.

According to specific embodiments, the disease is not Bardet-Biedl syndrome or in comorbidity with Bardet-Biedl syndrome.

As used herein, the term “BBS”, refers to the expression product of a BBS gene identified by its association with Bardet-Biedl syndrome (OMIM 209900). Mutations in a BBS gene leading to loss or dysfunction BBS result in Bardet-Biedl syndrome phenotype.

According to specific embodiments, BBS is human BBS.

According to specific embodiments, the BBS is selected from the group consisting of BBS1 (Gene ID 582), BBS2 (Gene ID 583), BBS3 (Gene ID 84100) BBS4 (Gene ID 585), BBS5 (Gene ID 129880), BBS6 (Gene ID 8195), BBS7 (Gene ID 55212), BBS8 (Gene ID 123016), BBS9 (Gene ID 27241), BBS10 (Gene ID 79738), BBS11 (Gene ID 22954), BBS12 (Gene ID 166379), BBS13 (Gene ID 54903), BBS14 (Gene ID 80184), BBS15 (Gene ID 51057), BBS16 (Gene ID 10806), BBS17 (Gene ID 54585), BBS18 (Gene ID 92482), BBS19 (Gene ID 11020), BBS20 (Gene ID 80173) and BBS21 (Gene ID 157657), each possibility represents a separate embodiment of the present invention.

According to specific embodiments, the BBS is selected from the group consisting of BBS1, BBS2, BBS3, BBS4, BBS5, BBS6, BBS7, BBS8, BBS9, BBS10, BBS11, BBS13, BBS14, BBS15, BBS16, BBS17, BBS18, BBS19, BBS20 and BBS21.

According to specific embodiments, the BBS is selected from the group consisting of BBS1, BBS2, BBS4, BBS5, BBS7, BBS8, BBS9 and BBS18.

According to specific embodiments, BBS is BBS4 (Gene ID 585).

According to a specific embodiment, the BBS4 refers to the human BBS4, such as provided in the following Accession Numbers: NM_001252678, NM_033028, NM_001320665, NP_001239607, NP_001307594, NP_149017, or a homolog or ortholog thereof.

According to specific embodiments, BBS is not BBS12 (Gene ID 166379).

As used herein the phrase “downregulates expression and/or activity” refers to downregulating the expression of BBS at the genomic (e.g. homologous recombination and site specific endonucleases) and/or the transcript level using a variety of molecules which interfere with transcription and/or translation (e.g., RNA silencing agents) or on the protein level (e.g., aptamers, small molecules and inhibitory peptides, antagonists, enzymes that cleave the polypeptide, antibodies and the like).

The expression and/or activity is generally expressed in comparison to the expression and/or activity in a cell of the same species but not contacted with the agent or contacted with a vehicle control, also referred to as control.

According to specific embodiments, down regulating expression and/or activity refers to a decrease of at least 5% in expression and/or activity in the presence of the agent in comparison to same in the absence of the agent, as determined by e.g. PCR, ELISA, Western blot analysis, immunoprecipitation, flow cytometry, immuno-staining. According to a specific embodiment, the decrease is in at least 10%, 30%, 40% or even higher say, 50%, 60%, 70%, 80%, 90% or even 100%. According to specific embodiments, the decrease is at least 1.5 fold, at least 2 fold, at least 3 fold, at least 5 fold, at least 10 fold, or at least 20 fold as compared to same in the absence of the agent.

According to specific embodiments, down regulating expression refers to the absence of mRNA and/or protein, as detected by RT-PCR or Western blot, respectively.

According to specific embodiments, downregulating activity comprises affecting localization of BBS.

Thus, according to specific embodiments, down regulating activity is effected by inhibiting localization of BBS to the ER [e.g. by binding and/or modifying an ER localization signal (ELS)].

Down regulation of expression and/or activity may be either transient or permanent.

According to a specific embodiment the agent specifically downregulates BBS and not an activator or effector thereof.

According to a specific embodiment the agent specifically binds BBS.

Non-limiting examples of agents capable of down regulating BBS expression are described in details hereinbelow. Measures should be taken to use molecules that penetrate the cell membrane or modified to enter through the cell membrane.

Down-Regulation at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, downregulation of BBS can be achieved by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g., the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs, siRNAs, miRNAs, shRNAs and antisense.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g., BBS4) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is suggested to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the BBS mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www(dot)ambion(dot)com/techlib/tn/91/912.html).

Second, potential target sites are compared to an appropriate genomic database (e.g., human, mouse, rat etc.) using any sequence alignment software, such as the BLAST software available from the NCBI server (www(dot)ncbi(dot)nhm(dot)nih(dot)gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

A non-limiting Example of BBS4 siRNA is provided in SEQ ID NOs: 45-46.

A non-limiting Example of BBS6 siRNA is provided in SEQ ID NOs: 49-50, as described in e.g. Kim J C, et al. J Cell Sci. 2005; 118(Pt 5):1007-1020.

RNA silencing agent suitable for use with some embodiments of the invention can also be designed and obtained commercially from e.g., Origene (see e.g. www(dot)origene(dot)com/category/rnai?q=RNAi+and+BBS4).

It will be appreciated that, and as mentioned hereinabove, the RNA silencing agent of some embodiments of the invention need not be limited to those molecules containing only RNA, but further encompasses chemically-modified nucleotides and non-nucleotides.

miRNA and miRNA mimics—According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” or “miRNA mimic” refers to synthetic non-coding RNAs that are capable of entering the RNAi pathway and regulating gene expression. miRNA mimics imitate the function of endogenous miRNAs and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-0,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

Preparation of miRNAs mimics can be effected by any method known in the art such as chemical synthesis or recombinant methods.

It will be appreciated from the description provided herein above that contacting cells with a miRNA may be effected by transfecting the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a BBS can be effected using an antisense polynucleotide capable of specifically hybridizing with an mRNA transcript encoding BBS.

Design of antisense molecules which can be used to efficiently downregulate a BBS must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Antisense suitable for use with some embodiments of the invention can also be designed and obtained commercially from e.g., Origene (see e.g. www(dot)origene(dot)com/category/mai?q=RNAi+ and+BBS4).

Nucleic acid agents can also operate at the DNA level as summarized infra.

Downregulation of BBS can also be achieved by inactivating the gene via introducing targeted mutations involving loss-of function alterations (e.g. point mutations, deletions and insertions) in the gene structure.

As used herein, the phrase “loss-of-function alterations” refers to any mutation in the DNA sequence of a gene (e.g., BBS4) which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments loss-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene. In such instances the e.g. BBS4 may be in a homozygous form or in a heterozygous form. According to this embodiment, homozygosity is a condition where both alleles at the e.g. BBS4 locus are characterized by the same nucleotide sequence. Heterozygosity refers to different conditions of the gene at the e.g. BBS4 locus.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases (e.g. Cre recombinase and Flp recombinase), PB transposases and genome editing by engineered nucleases (e.g. meganucleases, Zinc finger nucleases (ZFNs), transcription-activator like effector nucleases (TALENs) and CRISPR/Cas system). Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG family, the GIY-YIG family, the His-Cys box family and the HNH family. These families are characterized by structural motifs, which affect catalytic activity and recognition sequence. For instance, members of the LAGLIDADG family are characterized by having either one or two copies of the conserved LAGLIDADG motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8, 124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editorm genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fokl. Additionally Fokl has the advantage of requiring dimerization to have nuclease activity and this means the specificity increases dramatically as each nuclease partner recognizes a unique DNA sequence. To enhance this effect, Fokl nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fokl domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciencesm (Richmond, Calif.).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, Calif.).

CRISPR-Cas system—Many bacteria and archea contain endogenous RNA-based adaptive immune systems that can degrade nucleic acids of invading phages and plasmids. These systems consist of clustered regularly interspaced short palindromic repeat (CRISPR) genes that produce RNA components and CRISPR associated (Cas) genes that encode protein components. The CRISPR RNAs (crRNAs) contain short stretches of homology to specific viruses and plasmids and act as guides to direct Cas nucleases to degrade the complementary nucleic acids of the corresponding pathogen. Studies of the type II CRISPR/Cas system of Streptococcus pyogenes have shown that three components form an RNA/protein complex and together are sufficient for sequence-specific nuclease activity: the Cas9 nuclease, a crRNA containing about 20 base pairs of homology to the target sequence, and a trans-activating crRNA (tracrRNA) (Jinek et al. Science (2012) 337: 816-821.). It was further demonstrated that a synthetic chimeric guide RNA (gRNA) composed of a fusion between crRNA and tracrRNA could direct Cas9 to cleave DNA targets that are complementary to the crRNA in vitro. It was also demonstrated that transient expression of Cas9 in conjunction with synthetic gRNAs can be used to produce targeted double-stranded brakes in a variety of different species (Cho et al., 2013; Cong et al., 2013; DiCarlo et al., 2013; Hwang et al., 2013a,b; Jinek et al., 2013; Mali et al., 2013).

The CRIPSR/Cas system for genome editing contains two distinct components: a gRNA and an endonuclease e.g. Cas9.

The gRNA encodes a combination of the target homologous sequence (crRNA) and the endogenous bacterial RNA that links the crRNA to the Cas9 nuclease (tracrRNA) in a single chimeric transcript. The gRNA/Cas9 complex is recruited to the target sequence by the base-pairing between the gRNA sequence and the complement genomic DNA. For successful binding of Cas9, the genomic target sequence must also contain the correct Protospacer Adjacent Motif (PAM) sequence immediately following the target sequence. The binding of the gRNA/Cas9 complex localizes the Cas9 to the genomic target sequence so that the Cas9 can cut both strands of the DNA causing a double-strand break. Just as with ZFNs and TALENs, the double-stranded brakes produced by CRISPR/Cas can undergo homologous recombination or NHEJ.

The Cas9 nuclease has two functional domains: RuvC and HNH, each cutting a different DNA strand. When both of these domains are active, the Cas9 causes double strand breaks in the genomic DNA.

A significant advantage of CRISPR/Cas is that the high efficiency of this system coupled with the ability to easily create synthetic gRNAs enables multiple genes to be targeted simultaneously. In addition, the majority of cells carrying the mutation present biallelic mutations in the targeted genes.

However, apparent flexibility in the base-pairing interactions between the gRNA sequence and the genomic DNA target sequence allows imperfect matches to the target sequence to be cut by Cas9.

Modified versions of the Cas9 enzyme containing a single inactive catalytic domain, either RuvC- or HNH-, are called ‘nickases’. With only one active nuclease domain, the Cas9 nickase cuts only one strand of the target DNA, creating a single-strand break or ‘nick’. A single-strand break, or nick, is normally quickly repaired through the HDR pathway, using the intact complementary DNA strand as the template. However, two proximal, opposite strand nicks introduced by a Cas9 nickase are treated as a double-strand break, in what is often referred to as a ‘double nick’ CRISPR system. A double-nick can be repaired by either NHEJ or HDR depending on the desired effect on the gene target. Thus, if specificity and reduced off-target effects are crucial, using the Cas9 nickase to create a double-nick by designing two gRNAs with target sequences in close proximity and on opposite strands of the genomic DNA would decrease off-target effect as either gRNA alone will result in nicks that will not change the genomic DNA.

Modified versions of the Cas9 enzyme containing two inactive catalytic domains (dead Cas9, or dCas9) have no nuclease activity while still able to bind to DNA based on gRNA specificity. The dCas9 can be utilized as a platform for DNA transcriptional regulators to activate or repress gene expression by fusing the inactive enzyme to known regulatory domains. For example, the binding of dCas9 alone to a target sequence in genomic DNA can interfere with gene transcription.

There are a number of publically available tools available to help choose and/or design target sequences as well as lists of bioinformatically determined unique gRNAs for different genes in different species such as the Feng Zhang lab's Target Finder, the Michael Boutros lab's Target Finder (E-CRISP), the RGEN Tools: Cas-OFFinder, the CasFinder: Flexible algorithm for identifying specific Cas9 targets in genomes and the CRISPR Optimal Target Finder.

gRNAs suitable for use with some embodiments of the invention can also be designed and obtained commercially from e.g., Abm® (see e.g. www(dot)abmgood(dot)com/catalogsearch/result/?cat=5+&q=BBS4&utm_source=GeneCards&utm_medium=cpc&utm_campaign=CRISPR&utm_term=BBS4&utm_content=2) and Origene (see e.g. www(dot)origene(dot)com/catalog/gene-expression/knockout-kits-crispr/kn406210/bbs4-human-gene-knockout-kit-crispr).

In order to use the CRISPR system, both gRNA and Cas9 should be expressed in a target cell. The insertion vector can contain both cassettes on a single plasmid or the cassettes are expressed from two separate plasmids. CRISPR plasmids are commercially available such as the px330 plasmid from Addgene.

“Hit and run” or “in-out”—involves a two-step recombination procedure. In the first step, an insertion-type vector containing a dual positive/negative selectable marker cassette is used to introduce the desired sequence alteration. The insertion vector contains a single continuous region of homology to the targeted locus and is modified to carry the mutation of interest. This targeting construct is linearized with a restriction enzyme at a one site within the region of homology, electroporated into the cells, and positive selection is performed to isolate homologous recombinants. These homologous recombinants contain a local duplication that is separated by intervening vector sequence, including the selection cassette. In the second step, targeted clones are subjected to negative selection to identify cells that have lost the selection cassette via intrachromosomal recombination between the duplicated sequences. The local recombination event removes the duplication and, depending on the site of recombination, the allele either retains the introduced mutation or reverts to wild type. The end result is the introduction of the desired modification without the retention of any exogenous sequences.

The “double-replacement” or “tag and exchange” strategy—involves a two-step selection procedure similar to the hit and run approach, but requires the use of two different targeting constructs. In the first step, a standard targeting vector with 3′ and 5′ homology arms is used to insert a dual positive/negative selectable cassette near the location where the mutation is to be introduced. After electroporation and positive selection, homologously targeted clones are identified. Next, a second targeting vector that contains a region of homology with the desired mutation is electroporated into targeted clones, and negative selection is applied to remove the selection cassette and introduce the mutation. The final allele contains the desired mutation while eliminating unwanted exogenous sequences.

Site-Specific Recombinases—The Cre recombinase derived from the P1 bacteriophage and Flp recombinase derived from the yeast Saccharomyces cerevisiae are site-specific DNA recombinases each recognizing a unique 34 base pair DNA sequence (termed “Lox” and “FRT”, respectively) and sequences that are flanked with either Lox sites or FRT sites can be readily removed via site-specific recombination upon expression of Cre or Flp recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and Flp recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and Flp/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or Flp in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], Tol2 [Kawakami et al. PNAS (2000) 97 (21): 11403-11408] or Frog Prince [Miskey et al. Nucleic Acids Res. December 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas Tol2 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or Flp/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

Genome editing using recombinant adeno-associated virus (rAAV) platform—this genome-editing platform is based on rAAV vectors which enable insertion, deletion or substitution of DNA sequences in the genomes of live mammalian cells. The rAAV genome is a single-stranded deoxyribonucleic acid (ssDNA) molecule, either positive- or negative-sensed, which is about 4.7 kb long. These single-stranded DNA viral vectors have high transduction rates and have a unique property of stimulating endogenous homologous recombination in the absence of double-strand DNA breaks in the genome. One of skill in the art can design a rAAV vector to target a desired genomic locus and perform both gross and/or subtle endogenous gene alterations in a cell. rAAV genome editing has the advantage in that it targets a single allele and does not result in any off-target genomic alterations. rAAV genome editing technology is commercially available, for example, the rAAV GENESIS™ system from Horizon™ (Cambridge, UK).

It will be appreciated that the agent can be a mutagen that causes random mutations and the cells exhibiting downregulation of the expression level and/or activity of BBS may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

In addition, one ordinarily skilled in the art can readily design a knock-in/knock-out construct including positive and/or negative selection markers for efficiently selecting transformed cells that underwent a homologous recombination event with the construct. Positive selection provides a means to enrich the population of clones that have taken up foreign DNA. Non-limiting examples of such positive markers include glutamine synthetase, dihydrofolate reductase (DHFR), markers that confer antibiotic resistance, such as neomycin, hygromycin, puromycin, and blasticidin S resistance cassettes. Negative selection markers are necessary to select against random integrations and/or elimination of a marker sequence (e.g. positive marker). Non-limiting examples of such negative markers include the herpes simplex-thymidine kinase (HSV-TK) which converts ganciclovir (GCV) into a cytotoxic nucleoside analog, hypoxanthine phosphoribosyltransferase (HPRT) and adenine phosphoribosytransferase (ARPT).

Alternatively or additionally, downregulation of BBS can be achieved at the protein level.

Down-Regulation at the Polypeptide Level

According to specific embodiments, the agent which downregulates expression and/or activity of BBS is a small molecule or a peptide which binds and/or interferes with BBS protein activity.

According to specific embodiments, the agent which downregulates expression and/or activity of BBS is a molecule which binds to and/or cleaves BBS. Such molecules can be a small molecule, an inhibitory peptide.

Another agent which can be used along with some embodiments of the invention to downregulate BBS is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

According to specific embodiments the agent capable of downregulating BBS is an antibody or antibody fragment capable of specifically binding BBS. Preferably, the antibody specifically binds at least one epitope of BBS. As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

As BBS is localized intracellularly, an antibody or antibody fragment capable of specifically binding BBS is typically an intracellular antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

It will be appreciated that a non-functional analogue of at least a catalytic or binding portion of BBS can be also used as an agent which downregulates BBS.

Thus, according to specific embodiments, the agent is an aptamer, a peptide, a small molecule or an antibody.

According to specific embodiments, the agent is an aptamer, a peptide or a small molecule.

According to specific embodiments, the agent of the present invention can be administered to a subject in combination with other established (e.g. gold standard) or experimental therapeutic regimen to treat a disease associated with cells exhibiting ER stress and/or disease that can benefit from neural tissue formation or regeneration including, but not limited to analgesics, chemotherapeutic agents, radiotherapeutic agents, cytotoxic therapies (conditioning), hormonal therapy, antibodies, antibiotics, anti-inflammatory drugs and other treatment regimens (e.g., surgery) which are well known in the art.

Thus, according to another aspect of the present invention there is provided an article of manufacture comprising an agent which downregulates expression and/or activity of BBS and a therapeutic for treating a disease associated with cells exhibiting ER stress.

According to specific embodiments, the article of manufacture is identified for the treatment of a disease associated with cells exhibiting ER stress.

According to an additional or an alternative aspect of the present invention, there is provided an article of manufacture comprising an agent which downregulates expression and/or activity of BBS and a therapeutic for treating a disease a disease that can benefit from neural tissue formation or regeneration.

According to specific embodiments, the article of manufacture is identified for the treatment of a disease that can benefit from neural tissue formation or regeneration.

According to specific embodiments, the agent which downregulates expression and/or activity of BBS and the additional therapeutic are packaged in separate containers.

According to specific embodiments, the agent which downregulates expression and/or activity of BBS and the additional therapeutic are packaged in a co-formulation.

The agents and compounds of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent or the compound described herein accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent [e.g., production of a chimeric fusion protein that comprises a transport peptide that has an affinity for an endothelial cell surface molecule in combination with an agent that is itself incapable of crossing the blood brain barrier (BBB)] in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., a disease associated with cells exhibiting ER stress e.g. cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide levels of the active ingredient that are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to another aspect of the present invention there is provided a method of diagnosing a disease associated with cells exhibiting ER stress in a subject, the method comprising determining a level of expression and/or activity of BBS in a biological sample of the subject, wherein a level of expression and/or activity of BBS above a predetermined threshold in said sample is indicative of the disease associated with cells exhibiting ER stress.

As used herein the phrase “diagnosing” refers to classifying a pathology (i.e., a disease associated with cells exhibiting ER stress) or a symptom, determining a severity of the pathology, monitoring pathology progression, forecasting an outcome of a pathology and/or prospects of recovery.

Determining a level of expression and/or activity are known in the art, and may be effected on the RNA level (using techniques such as Northern blot analysis, RT-PCR and oligonucleotides microarray) and/or the protein level (using techniques such as ELISA, Western blot analysis, immunohistochemistry and the like, which may be effected using antibodies specific to the component).

According to specific embodiments, determining the level of expression and/or activity of BBS is effected in-vitro or ex-vivo.

Non-limiting examples of biological samples include but are not limited to, a cell obtained from any tissue biopsy, a tissue, an organ, body fluids such as blood, and rinse fluids.

According to specific embodiments, the extent of increase of the level of expression and/or activity from a predetermined threshold is derived from a control sample, such as a healthy control sample, a sample with a known disease state or a sample with a known ER stress extent.

Thus, the predetermined level can be experimentally determined by comparing BBS expression and/or activity in a biological sample of a healthy subject with BBS expression and/or activity in the same type of biological sample of a subject having a disease associated with cells exhibiting ER stress with known stage.

According to specific embodiments, the increase from a predetermined threshold is statistically significant.

According to specific embodiments, the increase from a predetermined threshold is at least 1.1 fold, at least 1.2 fold, at least 1.3 fold, at least 1.4 fold, at least 1.5 fold, at least 2 fold, at least 3 fold, at least 5 fold, at least 10 fold, or at least 20 fold as compared to the levels of expression and/or activity in a control sample.

According to specific embodiments, the increase from a predetermined threshold is at least 2%, at least 5%, at least 10%, at least 20%, at least 30%, at least 40%, at least 50%, at least 60%, at least 70%, at least 80%, at least 90%, e.g., 100%, at least 200%, at least 300%, at least 400%, at least 500%, at least 600% as compared the levels of expression and/or activity in a control sample.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods for Examples 1 and 2

Cell culture and Differentiation induction—Mouse 3T3F442A pre-adipocytes (ATCC) were grown under normal conditions (5% C02 at 37° C.). Cells were maintained in Dulbecco's Modified Eagle's Medium (DMEM, Gibco, USA) containing 10% bovine serum (BS, Biological Industries, Israel) and 1% penicillin-streptomycin (BI, Israel). Two days post-confluent adipocyte cells were induced to differentiate by differentiation medium, containing: DMEM, 10% fetal calf serum (FBS, Israel) and 1% penicillin-streptomycin. ER stress was induced by incubating cells with 5 μg/μl Tunicamycin (TM) for 6 hours or 2 μM Thapsigargin (TG) for 2 hours (Sigma, Israel).

Short-interfering RNA-mediated Knock-down—In order to knock down BBS4, constructs were designed to express short hairpin interfering RNA (shiRNA, SEQ ID NO: 45-46)

and were cloned by GenScript into p.RANT-H1/neo vector (Genscript, USA) previously⁽¹⁹⁾. Cells were transfected at 70-80% confluence using TransIT-LT1 transfection reagent (Mirus, USA) according to the manufacturer's protocol. 48 hours post transfection, medium was changed to selection medium containing 1.5 mg/ml of G418 (Gibco, USA). Empty vector was used as a control. Transfection efficiency was tasted by western blotting/RT-qPCR.

Generation of over expression BBS4 cells—For over expression of BBS4, 3T3-F442A preadipocytes were transfected both with pEF4/Hisc vector containing the BBS4 coding sequence [CDS, The full length cDNA of BBS4 was generated by PCR using primers: F: 5′-CATGGCTGAAGTGAAGCTTGG (SEQ ID NO: 47) and R: 5′-TC TCGGTTITCCTGTTTG (SEQ ID NO: 48)] and with the pEF4/Hisc empty vector as previously detailed (9). Twenty-four hours prior to transfection, cells were re-plated in six wells plates. Transfections were performed using TransIT-LT1 transfection reagent (Mirus, USA). Forty-eight hours post transfection the medium was changed to selection medium containing 1 mg/ml Zeocin (Invitrogen, USA). Over expression was verified by western blotting/RT-qPCR.

Protein extraction—Total proteins of cell lysate were extracted using lysis buffer (Promega, Israel). Briefly, cells were washed with cold PBS, and lysed for 20 minutes on ice in an appropriate volume of lysis buffer (3×10⁷ cell per 1 ml lysis buffer) containing phosphatase inhibitor cocktail (Sigma, Israel). Following, samples were centrifuge for 20 minutes at 14,000 g at 4° C. The supernatant was transferred to a fresh tube. For endoplasmic reticulum (ER) protein extraction, PBS-washed cell pellets were gently re-suspended in a 1×MTE buffer (270 mM D-mannitol, 10 mM Tris-base, 0.1 mM EDTA, PH 7.4, 1 mM PMSF). The suspensions were sonicated on ice three times for 10 seconds each, separated by 10 seconds rest intervals followed by centrifugation at 1,400 g for 10 minutes. At this stage, 100 μl of the supernatant was labeled as “total protein” and stored immediately at −80° C. The remaining supernatant was centrifuged for 20 minutes at 15,000 g, 4° C. Following centrifugation, the ER fractions (supernatants) were transferred to discontinuous sucrose gradient (1.3 M, 1.5 M, 2 M) and centrifuged using an ultra-centrifuge at 152,000 g, 4° C. for 70 minutes. At this state, 500 μl of supernatant was labeled as the cytoplasmic fractions and stored at −80° C. until subsequent analyses. 400-600 μl of the large band at the interface of the 1.3 M sucrose gradient layer was extracted using a 20-G needle and 1 ml syringe and transferred to sterile 11×60 mm polyallomer tube. Following ultracentrifugation for 45 minutes at 126,000 g, 4° C., the supernatants were decanted and discarded. The pellets were resuspended in 100 μl PBS×1, pH 7.4, labeled as the ER fractions and stored immediately at −80° C. Protein concentration was determined by standard Bradford assay.

Western blot analysis—Polyacrylamide gel electrophoresis, protein transfer, and western blotting were performed using standard laboratory techniques [Green M R., Sambrook J (2012) Molecular Cloning: A Laboratory Manual. 4^{th} ed. Cold Spring Harbor Laboratory Press]. Briefly, proteins were extracted and samples were mixed with SDS sample buffer and incubated for 5 minutes at 95° C. Thirty μg of whole cell lysate were loaded on a 15-10% SDS polyacrylamide gel. The following primary antibodies were used: mouse anti-BBS4 (Abcam, Israel), rabbit anti-XBP1 (Abcam, Israel), mouse anti-PD1 (Abcam, Israel), mouse anti-ATF6α (Santa Cruz, Israel), rabbit anti-P-IRE1α (Abcam, Israel), rabbit anti-CHOP (Santa Cruz, Israel), goat anti-actin (Santa Cruz, Israel), mouse anti-SREBP1 (abcam, Israel). The following secondary antibodies were used: goat anti-mouse IgG (Abcam, Israel), goat anti-rabbit IgG (Abcam, Israel), donkey anti-goat (Santa Cruz, Israel). Signals were visualized using Western EZ-ECL reagent (BI, Israel). Densitometry analyses of immunoblots were performed using image QuantTL software (GE life sciences, Pis-cataway, NJ, USA). All proteins were quantified relative to the housekeeping protein actin.

RNA Isolation and cDNA synthesis—Total RNA was isolated from cultures of 3T3-F442A cells using TRIZOL® Reagent (Invitrogen, Rhenium, Modi'in, Israel). RNA integrity was tested by agarose gel electrophoresis (0.8% w/v) with ethidium bromide staining. Total RNA was quantitated by UV absorption at 260 nm using a spectrophotometer (NanoDropND-1000UV-vis; NanoDrop Technologies, Wilmington, Del., USA) and reverse transcribed into cDNA using SuperScript H reverse transcriptase and oligo-dT primers (Invitrogen Rhenium, Modi'in, Israel). cDNA was further analyzed by real-time PCR.

Real-time Quantitative PCR—Transcript levels were determined by quantitative PCR (QPCR) using SYBR® Green PCR Master Mix (LifeTechnologies, Rhenium, Modi'in, Israel). Gene specific primers (Table I hereinbelow) were designed using the Primer Express Software (Life Technologies, Rhenium, Modi'in, Israel). The qPCR primer pairs were designed across exons to avoid false positive signals from potentially contaminating genomic DNA. Primer and cDNA concentrations were optimized (including melt curve analyses). Relative expression values for all the genes studied were normalized to the control housekeeping genes GAPDH and/or S18.

TABLE 1
Primers sequences

| Target Gene | |
|---|---|
| (m = mouse) | sequence (5′→3′) |
| m BBS4 | F: CCATAACCTGGGAGTGTGCT |
| | (SEQ ID NO: 1) |
| | R: TCGATGGCTTTATCCAGGTC |
| | (SEQ ID NO: 2) |
| m XBP-1 | F: ATTCTGACGCTGTTGCCTCT |
| (for | (SEQ ID NO: 3) |
| RT-qPCR) | R: AAAGGGAGGCTGGTAAGGAA |
| | (SEQ ID NO: 4) |
| m XBP-1 | F: TTACGAGAGAAAACTCATGGGC |
| (for | (SEQ ID NO: 5) |
| splicing | R: GGGTCCAACTTGTCCAGAATGC |
| assay) | (SEQ ID NO: 6) |
| m CHOP | F: CTGGAAGCCTGGTATGAGGAT |
| | (SEQ ID NO: 7) |
| | R: GCAGGGTCAAGAGTAGTGAAGGT |
| | (SEQ ID NO: 8) |
| mATF6α | F: GGCCAGACTGTTTTGCTCTC |
| | (SEQ ID NO: 9) |
| | R: CCCATACTTCTGGTGGCACT |
| | (SEQ ID NO: 10) |
| m Bcl-2 | F: CTGGGATGCCTTTGTGGAA |
| | (SEQ ID NO: 11) |
| | R: TCAAACAGAGGTCGCATGCT |
| | (SEQ ID NO: 12) |
| mCaspase3 | F: AGCTTGGAACGGTACGCTAA |
| | (SEQ ID NO: 13) |
| | R: CGTACCAGAGCGAGATGACA |
| | (SEQ ID NO: 14) |
| m BAX | F: AGTGTCTCCGGCGAATTGG |
| | (SEQ ID NO: 15) |
| | R: GTCCACGTCAGCAATCATCCT |
| | (SEQ ID NO: 16) |
| m S18 | F: TCTAGTGATCCCTGAGAAGT |
| | (SEQ ID NO: 17) |
| | R: ACGCCCTTAATGGCAGTGAT |
| | (SEQ ID NO: 18) |
| m GAPDH | F: GTATGACTCCACTCACGGCAA |
| | (SEQ ID NO: 19) |
| | R: CCATTCTCGGCCTTGACTGT |
| | (SEQ ID NO: 20) |

XBP-1 Splicing Assay—Amplification of XBP-1 transcripts was effected using PCR kit MyTaq DNA polymerase (Origolab, Israel) according to manufacturer's protocol. Following PCR, the XBP-1 fragment was incubated with the restriction enzyme Pst1 (Thermo) and the products were run on a 2% agarose gel.

Immunofluorescence—Cells were grown and differentiated on cover slips. Following fixation with 4% formaldehyde, cells were permeabilized with 0.1% TRITON™ X-100 and incubated in blocking solution (3% iNGS) for 1 hour followed by overnight incubation with a primary antibody followed by exposure to secondary antibodies coupled to Alexa Flour (Abcam, Israel). Nuclear labeling was performed with DAPI (Sigma, Israel). Immunofluorescence staining was visualized with Olympus/Confocal microscope. For quantification of positive cells, clusters were randomly selected from triplicates of 2-3 independent experiments and the average value SEM was determined.

Transmission electron microscopy—TEM—Cells grown on cover slips were washed twice in PBS for 10 minutes. Fixation was effected using 2.5% glutaraldehyde (EMS, USA), 1 mg/ml Ruthenium Red (Gurr, UK) in cacodylate buffer (TED pella, USA), pH 7.2 for 2 hours. Following, cell were washed twice for 10 minutes in 0.1 M cacodylate buffer and placed in 1% Osmium tetroxide, 1 mg/ml Ruthenium Red (Gurr, UK) in 0.1 M cacodylate buffer and washed again for 10 minutes in 0.1 M cacodylate buffer. Cells were dehydrated using increasing concentration of ethanol (EtOH; 30%, 50%, 70%, 90%, 100%) for 5 minutes each. Cells were kept at 4° C. in 70% ethanol until further processing. Following dehydration, cells were placed for 1 hour in propilin oxid and araldite in a 1:1 ratio. Cells were removed from the cover slips and transferred to bimcapsules and centrifuged at 2000 rpm. propilin oxid and araldite in a 1:1 ratio was removed and propilin oxid and araldite in a 1:2 ratio was added for 1 hour, followed by cells centrifugation at 2000 rpm. propilin oxid and araldite in a 1:2 ratio was removed and only araldite was added for 1 hour, followed by cells centrifugation at 2000 rpm. New araldite was added and the cells were centrifuged once more and kept at 60° C. for 24 hours for polymerization. Samples were cut using LEICA ULTRACUT UCT ultra microtome. Sections were contrasted with Uranyl acetate and lead citrate, placed on grids and viewed using a Jeol Jem 1230 microscope.

Statistical analysis—Results were collected from 3 independent experiments, each performed in triplicates. Data are expressed as mean±standard error (SD) or as average SEM, as indicated. Statistical analysis was performed using GraphPad Prism 7.0; comparisons using one-way analysis of variance (ANOVA). Statistical significance (p<0.05) of differences between treatment groups is presented by (*).

### Example 1

### BBS4 Expression and Localization is Responsive to Er Stress (Using Adipocytes as a Model)

To study the role of BBS4 in endoplasmic reticulum (ER) stress induced unfolding protein response (UPR), murine preadipocytes were subjected to ER stress using Tunicamycin (TM) during in-vitro adipogenesis.

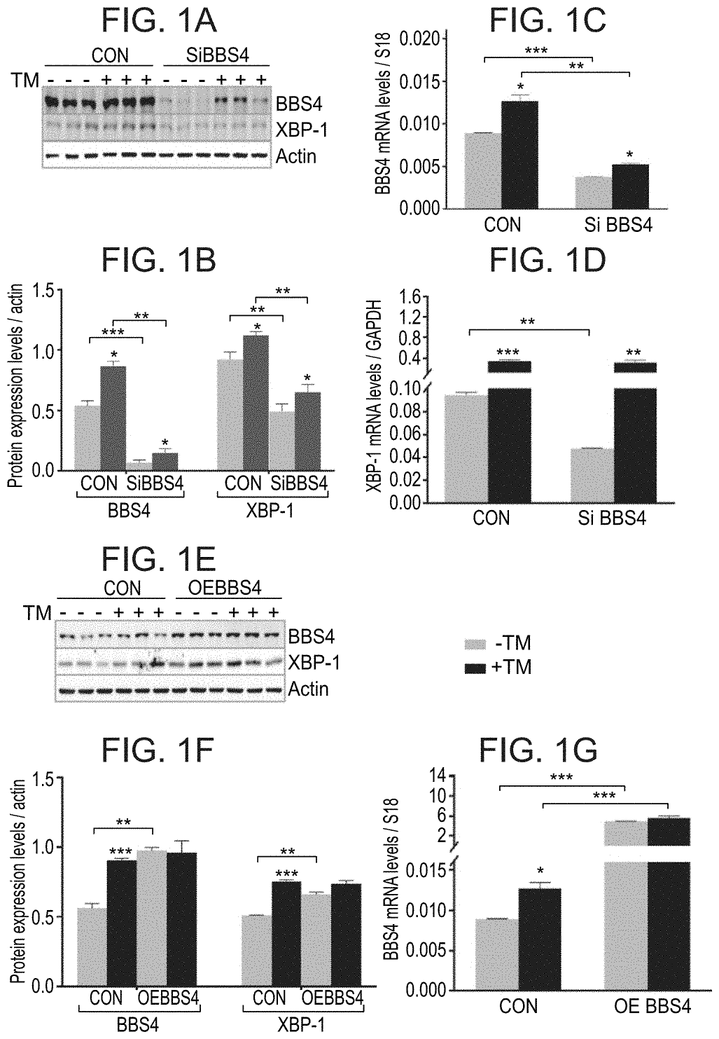

BBS4 expression is up-regulated under ER stress—Previous studies have shown that transcript levels of BBS4 (as well as other BBS genes) were significantly altered through adipocytes differentiation, reaching maximum levels at day 3⁽¹⁷⁾. TM induced ER stress resulted in a significant increase in BBS4 protein and transcript levels by 1.6 and 1.3 (P<0.05) fold, respectively, at day 3 of adipogenesis, as compared to un-treated control (FIGS. 1A-D). As expected, over-expression of BBS4 (OEBBS4) (FIGS. 1E-G) resulted in a significant elevation in both BBS4 protein (p<0.001) and transcript (p<0.05) levels by 400 and 1.8 fold, respectively, compared to control cells; while downregulation of BBS4 (SiBBS4) resulted in a significant reduction in both BBS4 protein and transcript levels (FIGS. 1A-C).

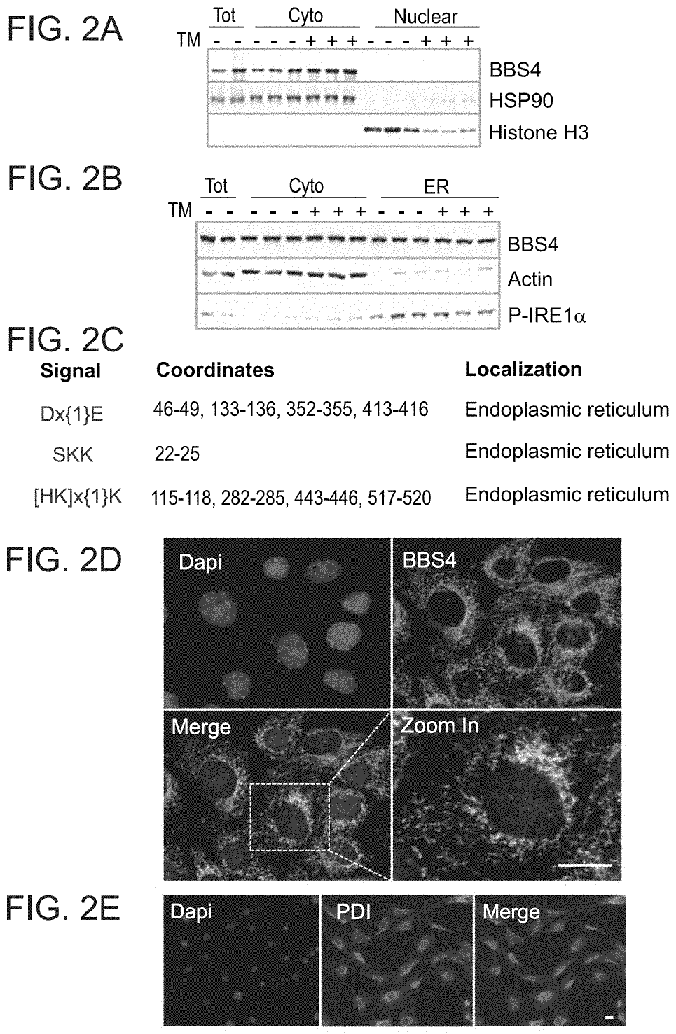

BBS4 is localized to the ER compartment—the subcellular localization of BBS4 was examined in adipoctes under TM-induced ER stress and control non-stressed conditions. Recently it was reported that BBS4 has a nuclear export signal (S), therefore, it was first hypothesized that BBS4 might have a role in ER factors that serve as transcription factors, thus related to nuclear function. However, while BBS4 protein was detected in the total and cytosol fractions following subcellular protein fractionation, the protein was not detected in the nuclear fraction either in the control nor in the TM-induced ER stress states (FIG. 2A). In the next step, in-silico studies using LocSigDB database⁽²⁹⁾ (genome(dot)unmc(dot)edu/LocSigDB) indicated that BBS4 has three predicted ER localization signals (ELS) (FIG. 2C). Using actin as a cytosolic marker and P-IRE1 a as an ER marker, it was shown using the protein subcellular fractionation that BBS4 is cellularly localized in the ER compartment, both in control and following TM treatment (FIG. 2B). In order to confirm this result, BBS4 cellular localization was studied by immunofluorescence labeling: As shown in FIGS. 2D-E, BBS4 was localized to the ER, as also affirmed by co-localization of BBS4 with the protein disulfide isomerase (PDI) which is a known ER marker, indicating and reinforcing that BBS4 cellular location in the ER.

### Example 2

### Down-Regulation of BBS4 Affects Er Stress Induced Unfolding Protein Response (Using Adipocytes as a Model)

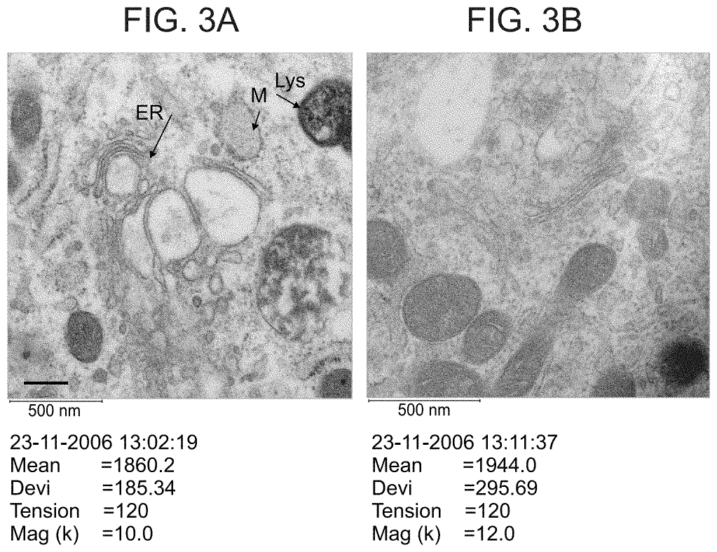

BBS4 silencing affects cells' morphology—TEM analysis of SiBBS4 adipocytes at day 8 of in-vitro differentiation demonstrated an exceptionally large amount of lysosomes and autophagic vacuoles containing cytoplasmic organelles in various states of autolysis (FIGS. 3A-B). These vacuoles, were described in differentiating 3T3-L1 cells as early as 1980 and may reflect the dramatic remodeling that accompanies differentiation. SiBB4 cells also contained more, larger in size and swollen ER indicative of ER stress compared to control cells (FIGS. 3A-B).

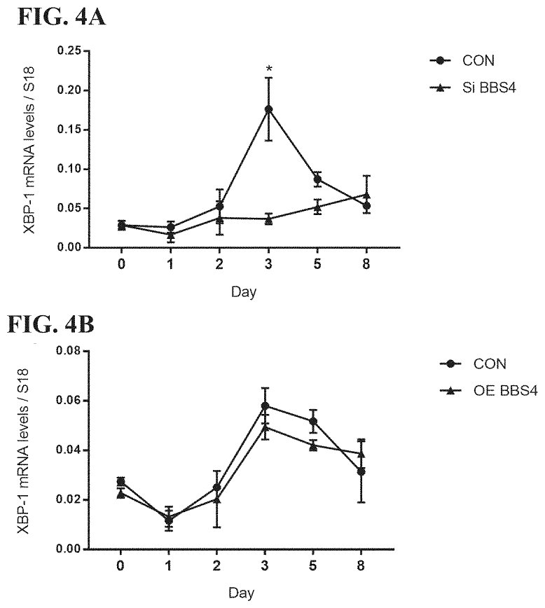

BBS4 silencing down-regulates XBP-1 expression levels—XBP-1 is crucial UPR transcription factor subjected to transcriptional and post-translational regulation. XBP-1 transcript levels during adipocytes differentiation (day 0, 1, 2, 3, 5, 8) was determined in control, SiBBS4 and OEBBS4 cells. In control adipocytes, XBP-1 transcript levels peak at day 3 of differentiation (FIG. 4A), corresponding to the peak of BBS4 transcript levels during adipocyte differentiation as previously reported [17]. On the contrary, in SiBBS4 cells XBP-1 mRNA levels were significantly (P<0.01) down-regulated at day 3 of differentiation compared to control cells (FIG. 4A).

In control cells TM-induced ER stress, resulted in a significant elevation in XBP-1 protein and transcript levels by 1.2 (P<0.05) and 3.5 (P<0.001) fold, respectively, compared to un-treated control cells, at day 3 of differentiation (FIGS. 1A-B and D). In SiBBS4 cells not treated with TM, XBP-1 protein and transcript levels were significantly reduced by 1.9 and 2 fold, respectively, compared to control adipocytes, at day 3 of differentiation (FIGS. 1A, B and D). Although XBP-1 levels in SiBBS4 cells were low, TM treatment of SiBBS4 cells resulted in a significant up-regulation of XBP-1 protein and transcript levels by 1.3 (P<0.05) and 6.5 (P<0.01) fold, respectively, compared to un-treated SiBBS4 cells (FIGS. 1A, B and D). It should be emphasized that though overexpression of BBS4 in OEBBS4 cells rescued the effect shown in SiBBS4 cells, exhibiting significant elevation in XBP-1 protein levels compared to control cells; under ER stress conditions overexpression of BBS4 did not have a significant effect on XBP-1 levels (FIGS. 1E-F and 4B).

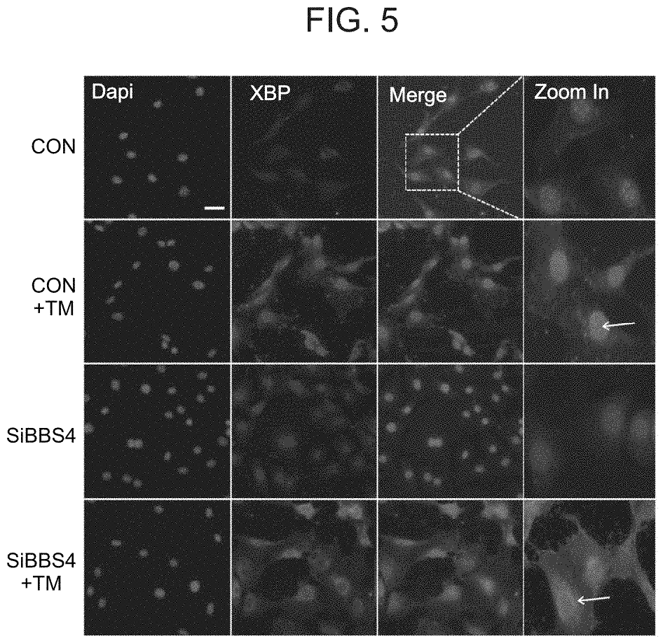

In order to determine whether XBP-1 subcellular pattern and transport was compromised due to the down regulation of XBP-1 in SiBBS4 cells, XBP-1 subcellular localization in SiBBS4 cells was analyzed in normal and TM-induced states using immunocytochemistry. As shown in FIG. 5, in both untreated control and SiBBS4 cells XBP-1 was located in the cytoplasm. Following TM-induced ER stress, XBP-1 was translocated to the nucleus in the control cells; however, in SiBBS4 cells XBP-1 was located in the cytoplasm and intensely accumulated (a ring shape-white arrow in FIG. 5) around the nucleus. These results indicate that XBP-1 is not transferred to the nucleus in SiBBS4 cells following TM-induced ER stress as expected and is retained in the ER.

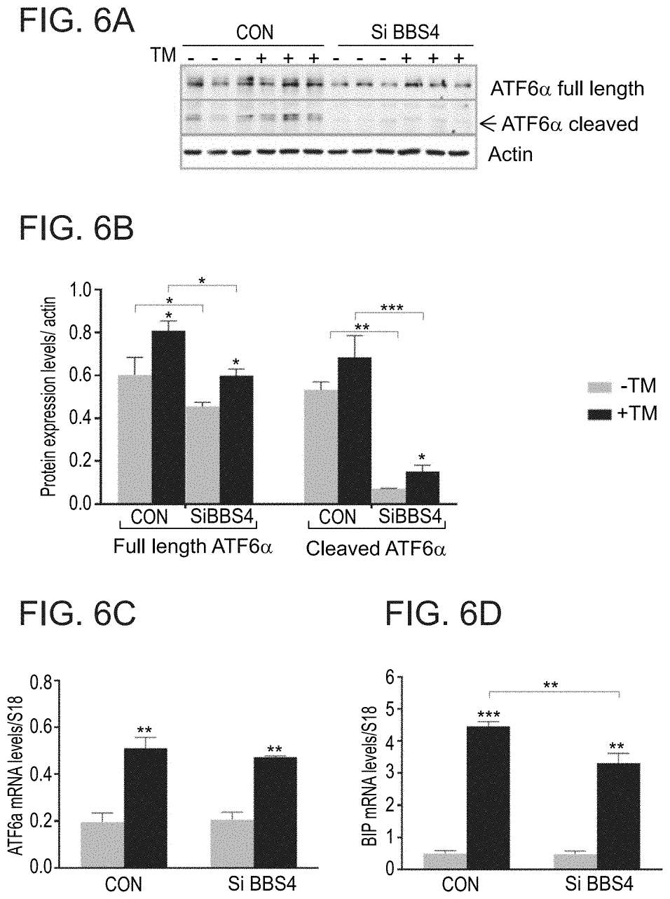

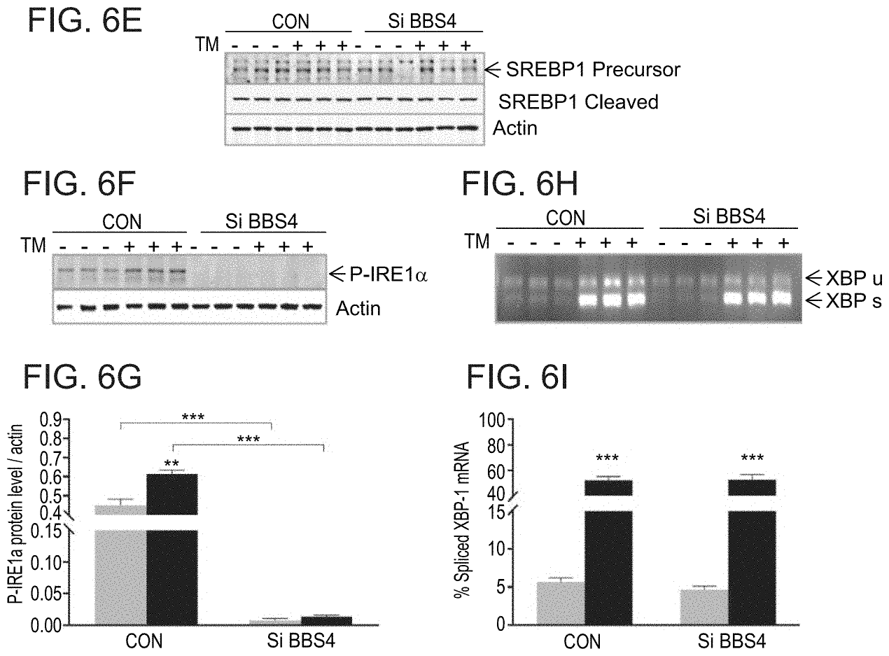

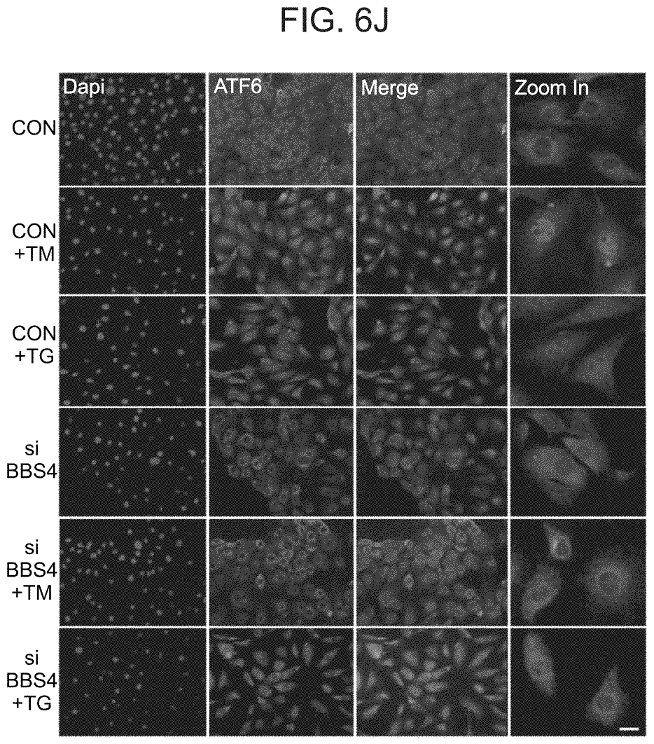

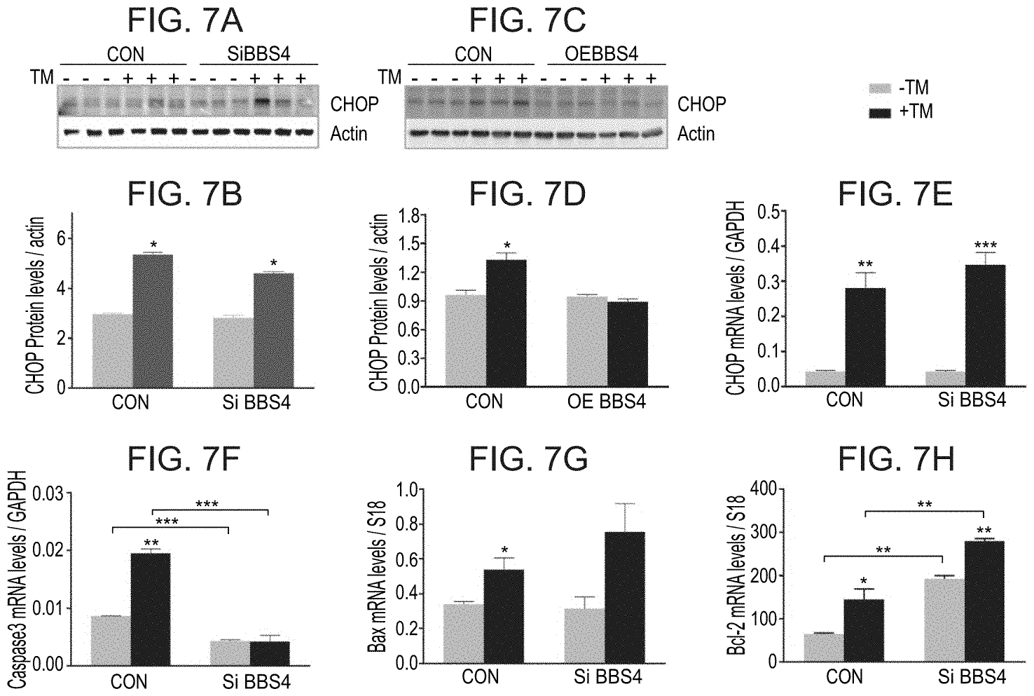

XBP-1 down regulation in SiBBS4 cells occurs due to specific inhibition of ATF6α and IRE1α—The transcription factor ATF6α is a major regulator of the UPR genes and has a direct transcript effect on XBP-1. Upon ER stress and UPR activation full length ATF6α is cleaved and translocated to the nucleus to act as transcription factor. Transcript levels of ATF6α were significantly (p<0.01) increased in response to TM treatment in control (by 2.6 fold) and in SiBBS4 cells (by 2.3 fold) (FIG. 6C). TM-induced ER stress resulted in a significant elevation in full length ATF6α protein levels both in control and SiBBS4 cells compared to untreated control and SiBBS4 untreated cells (by 1.4 and 1.3, respectively) (FIGS. 6A-B). This was also demonstrated in the cleaved activated ATF6α protein levels in control cells. Cleaved activated ATF6α levels were significantly reduced in SiBBS4 cells compared to control cells both in untreated and treated cells (FIGS. 6A-B) by 7.4 and 4.7 fold, respectively.

Given that the cleaved form of ATF6α is absent in SiBBS4 cells, the subcellular localization of ATF6α was visualized by immunofluorescence labeling (FIG. 6J). Without ER stress induction, the ATF6α is mainly located in the ER. Following ER stress [TM or Thapsigargin (TG)] the ATF6α is activated by dissociation from the GRP78 (BIP) and translocation to the Golgi apparatus, where it is cleaved into an active form, also known as cleaved ATF6α. The cleaved ATF6α migrates to the nucleus where it regulates expression of UPR genes. In order to insure that the specific TM mode of action through inhibition of ER protein glycosylation, is not the cause for the absence of cleaved ATF6α in SiBBS4 cells (FIGS. 6A-B), the cells were treated with another ER stress inducer, Thapsigargin (TG), which act through a different mechanism for ER stress induction (by inhibition of the SERCA pumps). In control cells, in correlation with the results obtained with TM, TG treatment resulted in accumulation of cleaved ATF6α form in the nucleus. Similarly to TM induced stress, following TG treatment ATF6α did not translocate to the nucleus and accumulated and retained in the ER compartment in SiBBS4 cells indicating that the full length ATF6α does not undergo through the normal process and that the defective process of ATF6α cleavage occurs due to BBS4 depletion and not due to inhibition of glycosylation (FIG. 6J). Dissociation from GRP78 (BIP) allows ATF6α to translocate to the Golgi apparatus via COPII vesicles, whereby the cleavage occurs followed by migration to the nucleus to transduce expression of UPR genes and ER chaperones such as GRP78 (BIP) [26-27]. In accordance with the reduction in cleaved ATF6α in SiBBS4 cells under TM-induced ER stress, GRP78 (BIP) transcript levels were significantly (P<0.01) down-regulated by 1.5 fold compared to control treated cells, further indicating the reduction in transducing action of cleaved ATF6α (FIG. 6D).

In the next step, the levels of phosphorylated IRE1α RNase (pIRE1α), which splices the XBP-1 mRNA, were analyzed. As expected, in response to TM-induced ER stress pIRE1α levels were significantly (P<0.01) upregulated by 1.2 fold in control cells. Significant (P<0.001) reduction in pIRE1α levels by 5 fold were found in SiBBS4 cells following TM treatment and also in untreated cells compared to control cells (FIGS. 6F-G). pIRE1α splices XBP-1, thus XBP-1 splicing was studied in non-ER stress and TM-induced ER stress conditions in both control adipocytes and SiBBS4 cells. TM-induced conditions significantly elevated XBP-1 splicing, however, the % of XBP splicing, both in non-ER stress and TM-induced conditions were not significantly different (FIGS. 6H-I).

Taken together, BBS4 protein and transcript levels are significantly up-regulated in differentiating adipocytes following TM-induced ER stress indicating responsiveness of BBS4 to ER stress. Furthermore, BBS4 is localized to the ER at day 3 of adipogenesis and participates in UPR activation through ATF6α and IRE1α regulation. BBS4 depletion in adipocytes results in depletion of cleaved ATF6α and consequently of IRE1α phosphorylation and XBP-1 reduction.

### Materials and Methods for Examples 3 and 4

Cell Culture and differentiation—Human SH-SY5Y Neuroblastoma Cell Line (ATCC) were grown on cell culture plates (Greiner Bio-one, Austria) as a sub-confluent culture in Dulbecco's modified Eagle's medium (DMEM, Gibco, USA) supplemented 10% fetal calf serum (FBS, BI, Israel) and 1% penicillin-streptomycin (BI, Israel). For differentiation, SH-SY5Y were incubated in low-serum medium (Dulbecco's modified Eagle's medium containing 1% FBS, and 1% penicillin-streptomycin) supplemented with retinoic acid (RA) to a final concertation of 10 μM. All cell lines were cultured in an atmosphere of 5% C02 at 37° C. ER stress was induced by incubating cells with 5 μg/μl Tunicamycin (TM), (Sigma-Aldrich, Israel) for 6 hours. Cell survival was studied after 24 hours of TM treatment using trypan blue staining (Sigma-Aldrich, Israel). Specifically, cells were trypsinized and stained with trypan blue solution, Trypan blue-negative viable cells and trypan blue—positive dead cells were counted under a light microscopy.

Rat PC-12 Pheochromocytoma Cell Line (ATCC) were grown on cell culture plates (Greiner Bio-one, Austria) as a sub-confluent culture in Dulbecco's modified Eagle's medium (DMEM, Gibco, USA) supplemented with 10% horse serum (HS, Biological Industries, Israel), 5% fetal calf serum (FBS, BI, Israel) and 1% penicillin-streptomycin (BI Israel). For differentiation, PC-12 cells were incubated in differentiation medium containing DMEM, 1% HS, and 1% penicillin-streptomycin and nerve growth factor (NGF) (50 ng/ml).

Short-interfering RNA-mediated Knock-down—Constructs were designed to express short hairpin interfering RNA (shiRNA, SEQ ID NO: 45-46), and were cloned by GenScript into constitutively-expressing GFP p.RANT-H1/neo vector (Genscript, USA) (described in Heon, E. et al. (2016) Human molecular genetics, 25(11), pp. 2283-2294). SH-SY5Y cells were transfected at 70-80% confluence using TransIT-LT1 transfection reagent (Mirus, USA) according to manufacture protocol. 48 hours following transfection, the cells were incubated in selection medium containing 1.5 mg/ml G418 (Gibco, USA). Empty vector was used as a negative control. Transfection efficiency was verified by RT-qPCR and western blot. Successfully transfected SH-SY5Y cells were sorted using fluorescence-activated cell sorting (FACS).

Fluorescence Activated Cell Sorting (FACS)—Sorting for siBBS4-GFP-expressing SY5Y cells was effected using a Maestro 2 Fluorescence Filter Sets system (Becton-Dickenson). Normal control cells were used to set the background level of fluorescence. Transfected cells were analyzed for fluorescence intensity and compared to control cells. Following, GFP-expressing cells were sorted by Sony SH800 FACS cell sorter using a 100 μm chip and a GFP filter set (525/50).

RNA Isolation and cDNA synthesis—Total RNA was isolated from SH-SY5Y cell cultures using TRI reagent (Trizol, Rhenium, Israel) according to the manufacturer's protocol. RNA was quantified by UV absorption using a spectrophotometer (UV-Vis spectrophotometer; NanoDrop 2000c Thermo Scientific, USA) and RNA integrity was tested by agarose gel electrophoresis (1%) with Ethidium Bromide (Mercury, Israel) staining. cDNA was synthesized using reverse transcriptase (Bioline, Israel) according to manufacturer's protocol and further analyzed by Real time PCR.

Real time quantitative PCR (gPCR)—Transcript levels were determined by quantitative PCR (qPCR) using SYBER green PCR Master Mix (Life Technologies, Rhenium) according to the manufacturer's instructions. Gene specific primers (Table 2 hereinbelow) were designed using the Primer 3 online program (bioinfo(dot)ut(dot)ee/primer3-0.4.0/) and purchased from Sigma-Aldrich (Rehovot, Israel). All primers were designed across exons, to prevent false negative results. cDNA and Primers concentrations were optimized (including melting curve analysis). Reactions were carried out using MxPro3000 apparatus (Stratagene, Santa Clara, Calif.) according to manufacturer's instructions. GAPDH was used for mRNA level normalization.

TABLE 2
Primers sequences

| Target Gene | |
|---|---|
| (h = human, | |
| r = rat) | sequence (5′ 3′) |
| r BBS4 | F: TGAGGAGAAGCTTGGGATGAAA |
| | (SEQ ID NO: 21) |
| | R: GCCCTGAGTCTCCTGAAGC |
| | (SEQ ID NO: 22) |
| r GAPDH | F: TGAGGAGAAGCTTGGGATGAAA |
| | (SEQ ID NO: 23) |
| | R: GCCCTGAGTCTCCTGAAGC |
| | (SEQ ID NO: 24) |
| h BBS4 | F:TCAAGCAGGTGGCCAGATCT |
| | (SEQ ID NO: 25) |
| | R: GGTTATGGCTGATCTCCCAATC |
| | (SEQ ID NO: 26) |
| h XBP1 | F: TGCTGAGTCCGCAGCAGGTG |
| | (SEQ ID NO: 27) |
| | R: GCTGGCAGGCTCTGGGGAAG |
| | (SEQ ID NO: 28) |
| hsXBP-1 | F: GGAGTTAAGACAGCGCTTGG |
| (for splicing | (SEQ ID NO: 29) |
| assay) | R: ACTGGGTCCAAGTTGTCCAG |
| | (SEQ ID NO: 30) |
| h CHOP | F: CTGCAAGAGGTCCTGTCTTC |
| | (SEQ ID NO: 31) |
| | R: CAATCAGAGCTCGGCGAGTC |
| | (SEQ ID NO: 32) |
| h ATF6 | F: GCCTTTATTGCTTCCAGCAG |
| | (SEQ ID NO: 33) |
| | R: TGAGACAGCAAAACCGTCTG |
| | (SEQ ID NO: 34) |
| h Nestin | F: GTAGCTCCCAGAGAGGGGAA |
| | (SEQ ID NO: 35) |
| | R: CTCTAGAGGGCCAGGGACTT |
| | (SEQ ID NO: 36) |
| Bax | F: GATGCGTCCACCAAGAAG |
| | (SEQ ID NO: 37) |
| | R: AGTTGAAGTTGCCGTCAG |
| | (SEQ ID NO: 38) |
| Bcl-2 | F: GAACTGGGGGAGGATTGTGG |
| | (SEQ ID NO: 39) |
| | R: ACTTCACTTGTGGCCCAGAT |
| | (SEQ ID NO: 40) |
| Caspase-3 | F: CTCTGGTTTCGGTGGGTGT |
| | (SEQ ID NO: 41) |
| | R: TCCAGAGTCCATTGATTCGCT |
| | (SEQ ID NO: 42) |
| h GAPDH | F: CCATGGGGAAGGTGAAGGTC |
| | (SEQ ID NO: 43) |
| | R: AGTGATGGCATGGACTGTGG |
| | (SEQ ID NO: 44) |

XBP-1 Splicing Assay—Amplification of sXBP-1 transcripts was effected using a PCR kit MyTaq DNA Polymerase (Bioline, Israel) according to the manufacturer's instructions. PCR products were run on 3% agarose gel or stored at −20° C. for further analysis.

Protein Extraction—Proteins were extracted from cells using lysis buffer (Progma, Israel). Briefly, cells were washed with cold PBS and lysed in an appropriate volume of lysis buffer containing phosphatase inhibitor cocktail (Sigma, Israel) for 20 minutes on ice. Following, samples were centrifuged for 20 minutes at 14,000 g at 4° C. The supernatant was transferred to a fresh tube and stored at −80° C. Protein concentration was measured using Bradford assay.

Western Blot—Equal concentrations of protein samples were mixed with 4×SDS sample buffer for a final volume of 30 μl and incubated for 5 minutes at 95° C. The samples were loaded on a 10-15% SDS polyacrylamide gel followed by transfer to a nitrocellulose membrane. Blocking was effected by incubation with 5% albumin for 1 hour. Following, the membrane was incubated overnight at 4° C. with a primary antibody. Blots were washed with Tris buffer saline with tween 20 (TBST) and incubated for 1 hour with a secondary antibody followed by 4 washes, 5 minutes each, with TBST. The antibodies used are listed in Table 3 hereinbelow. Blot detection was effected by 2 minutes incubation with EZ-ECL chemiluminescence detection kit (EZ-ECL kit, Biological industries, Israel). Densitometry analysis of immunoblots was performed using ImageJ software version1.4. All proteins were quantified relative to housekeeping protein (HSP90/Actin).

Immunofluorescence—Cells were grown on coverslip, fixed for 10 minutes with 4% formaldehyde in room temperature and washed twice with PBS. Following, cells were permeabilized with 0.1% triton-X-100 and incubated for 1 hour in blocking solution (3% iNGS) followed by over-night incubation with a primary antibody and exposure to a secondary antibody coupled to Alexa Flour (Abcam, Israel). The antibodies used are listed in Table 3 hereinbelow. Nuclear labeling was performed with DAPI (Sigma, Israel). Immunofluorescence staining was visualized using Olympus microscope and cells representing different cell clusters were randomly selected (quadruplet for each experiment).

TABLE 3
Antibodies used in western blot and immunofluorescence analyses

| Antibody | Dilution | Manufacturer |
|---|---|---|
| Anti-BBS4 | 1:1000 | Abcam |
| Anti-XBP-1 | 1:1000 | Abcam |
| Anti-CHOP | 1:500 | Abcam |
| Anti-ATF6 | 1:500 | Santz-Cruz |
| Anti-Nestin | 1:1000 | Abcam |
| Anti-HSP90 | 1:1000 | Abcam |
| Anti-Actin | 1:200 | Santz-Cruz |
| Goat anti Mouse IgG HRP Conjugate | 1:2000 | Abcam |
| Goat anti Rabbit IgG HRP Conjugate | 1:2000 | Abcam |
| Alexa Fluor 555 | 1:200 | Abcam |
| Alexa Fluor 488 | 1:200 | Abcam |

Differentiation assessment—Neuronal differentiation evaluation was performed using the neurite outgrowth assessment standard technique (Zhou, Lihan, et al., 2010), with small modifications. Briefly, cells went through differentiation and observed under confocal microscope at different time points during the differentiation process. Cells bearing at least one neurite with the length equivalent to the cell bodies considered to be differentiated and were scored at the indicated time points by independent observers. More than 400 cells from three different fields were counted per well.

Morphological differences during differentiation between control and siBBS4 cells were investigated using fluorescence confocal microscope (LSM 700 Zeiss). Control and siBBS4 cells were seed on 6 wells cell culture plates and differentiated using with NGF (50 ng/ml) for PC-12 and 10 μM RA for SH-SY5Y. Cells were derived from 2 independent experiments and were randomly selected for each treatment and randomly photographed at the indicated time points. Representative images for all samples are presented.

Cell Counting—PC-12 and SH-SY5Y cells were collected using Trypsin-EDTA 0.25% (Biological Industries, Israel) into fresh medium, centrifuged and re-suspended in 1 ml medium. 10 μl of cell sample were mixed with 10 μl Trypan blue solution 0.5% (Biological Industries, Israel), loaded on hemocytometer (Marienfeld, Germany) and counted using light microscopy.

Migration assay—wound healing assay (“scratch”)—The scratch wound assay was used to measure cell migration. The procedure described by Rodriguez et al. was followed. Briefly, siBBS4 and control SH-SY5Y cells were seeded on 12 wells plates and grown to 100% confluence in complete growth medium. A linear wound “scratch” was created using 200 μl pipette tip. After washing the cultures twice with PBS, cells were immediately photographed (t=0) and a photo was taken every 15 minutes for 10 hours using the Olympus IX81 microscope (×10). The gap area was measured in pixels and the migration rate was measured in pixels per hour by ImageJ software.

### Example 3

### Down-Regulation of BBS4 Affects Er Stress Induced Unfolding Protein Response (Using Neuronal Cells as a Model)

Neural differentiation is characterized by early ER stress manifested by UPR activation. Hence, the role of BBS4 in UPR activation during in-vitro neural differentiation of human SH-SY5Y Neuroblastoma cell line was studied.

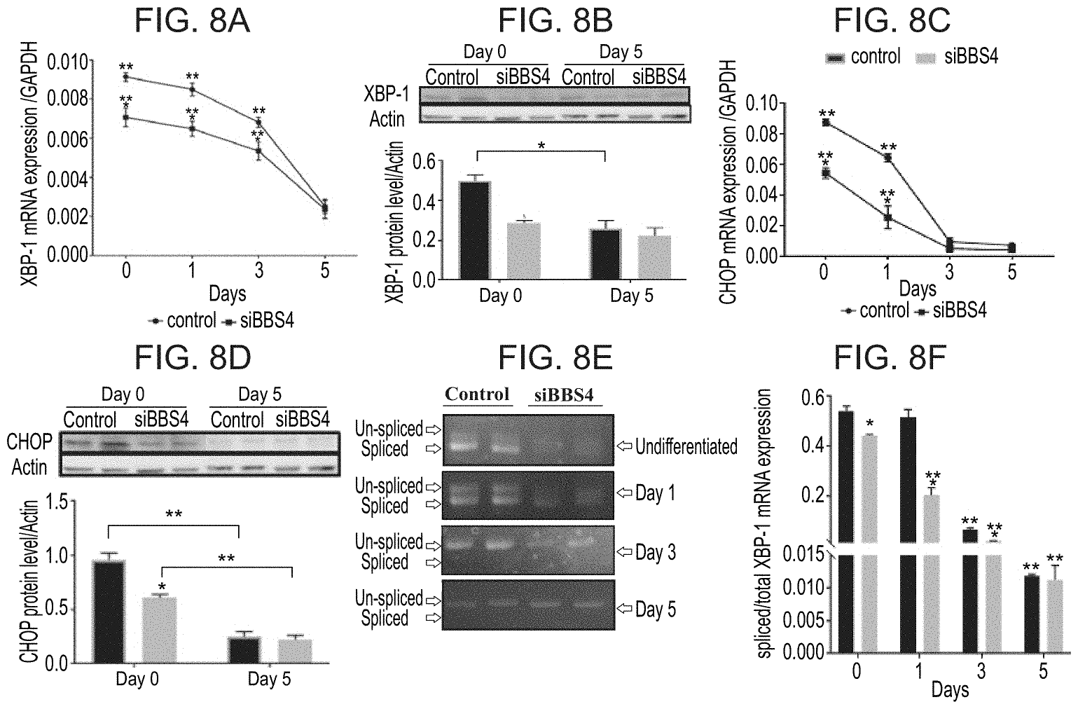

BBS4 silencing down-regulates XBP-1 expression, XBP-1 splicing and CHOP expression during neuronal differentiation—XBP-1 is crucial UPR transcription factor subjected to transcriptional and post-translational regulation. XBP-1 transcript levels during neuronal differentiation (day 0, 1, 3, 5) was determined in control and SiBBS4 cells. In both siBBS4 and control SH-SY5Y cells, XBP-1 transcript and protein levels were significantly (P<0.05) higher at early differentiation days (0-1 days), with significant reduction as differentiation progressed. In comparison to mature cells (day 5), XBP-1 levels in undifferentiated cells were significantly lower by 3.6-fold (transcript) and 1.9-fold (protein) in control cells, and by 3-fold (transcript) and 1.3-fold (protein) in siBBS4 cells. Importantly, XBP-1 levels at early differentiation days (days 0-3), were significantly (P<0.05) lower in SiBBS4 cells compared to control cells, by an average of 1.3-fold (transcript) and 1.7-fold (protein) (FIGS. 8A-B).

One of the hallmarks of activated UPR is XBP transcript splicing to sXBP-1, which serves as a transcription factor. The percentage of sXBP-1 was significantly (P<0.05) reduced by 39-fold and 45-fold between day 0 and day 5 in both siBBS4 and control cells, respectively, with parallel reduction in XBP-1 transcripts levels. Importantly, the percentage of sXBP-1 was significantly (P<0.05) lower in siBBS4 in comparison to the control cells throughout early differentiation (days 0-3) by an average of 2.6-fold, reaching a similar low levels at day 5 in both models (FIGS. 8E-F).

One of the branches of UPR is the PERK pathway, which plays an important role in ER stress-induced apoptosis. CHOP is a pro-apoptotic transcription factor regulating genes involved in either survival or death, such as the anti-apoptotic marker Bcl-2 (Puthalakath et al., 2007). CHOP transcript and protein levels were significantly (P<0.05) decreased during differentiation in both siBBS4 and control cells. In undifferentiated siBBS4 cells, CHOP levels were lower by 1.3-fold (transcript) and 2.7-fold (protein) compared to siBBS4 at day 5. Similarly, in undifferentiated control cells CHOP levels were lower by 12.4-fold (transcript) and by 3.8-fold compared to control cells at day 5. Following five days of differentiation, CHOP levels (transcript and protein) reached similar expression levels at in both siBBS4 and control cells (FIGS. 8C-D). However, during early differentiation (days 0-1) siBBS4 showed significantly (P<0.05) lower CHOP levels in comparison to control cells, by 1.6-fold and 1.5-fold (transcript and protein levels, respectively).

BBS4 silencing reduces UPR markers under TM-induced ER stress—siBBS4 undifferentiated SH-SY5Y cells demonstrated reduced ER stress by significant down-regulation of UPR markers (XBP-1, CHOP, % sXBP-1) levels compared to control SH-SY5Y cells. In the next step, the capability of siBBS4 SH-SY5Y cells to cope with induced ER stress (induced by treatment with TM for 6 hours) using UPR molecular markers including from all the three UPR branches (namely, ATF6α and BiP, CHOP, and XBP and sXBP).

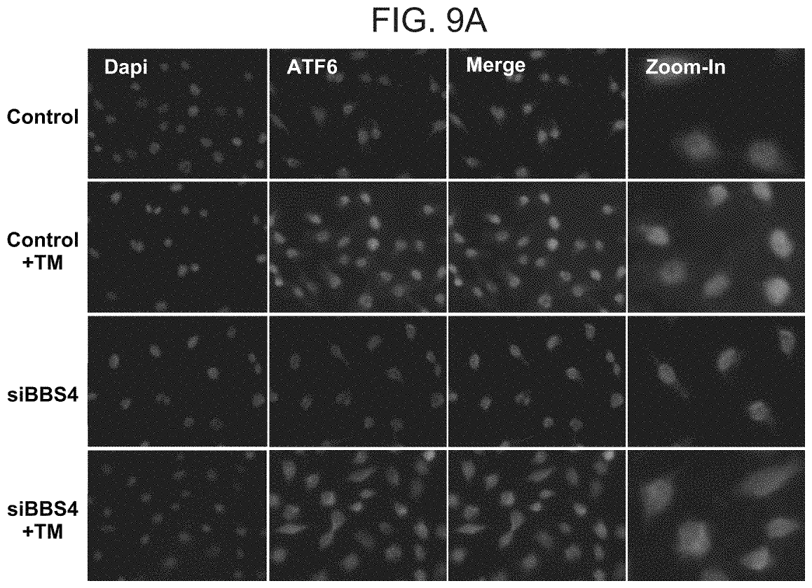

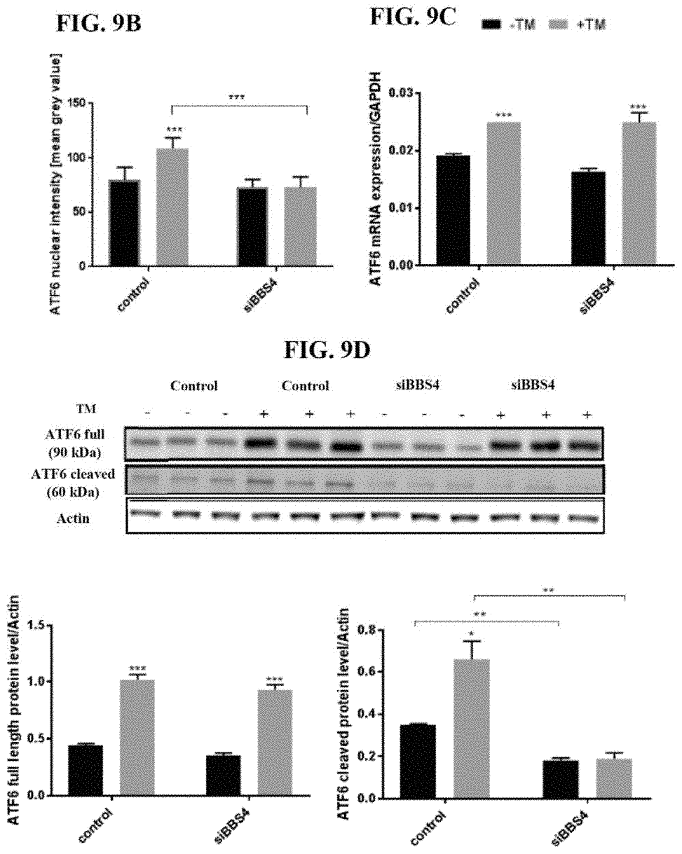

The transcription factor ATF6α is a major regulator of the UPR genes and has a direct transcript effect on XBP-1. Under naive conditions, ATF6α is bound to the ER membrane by GRP78 (BiP). Upon ER stimuli ATF6α dissociates from BiP and is transported to the Golgi for further processing by SIP and S2P, resulting in cleavage to form ATF6α p50 which translocates to the nucleus to act as transcription factor. To this end, the subcellular localization of ATF6α was examined in SH-SY5Y under TM-induced ER stress and control non-stressed conditions using immunohistochemistry (FIGS. 9A-B). As expected, in non-stressed siBBS4 and control SH-SY5Y cells, ATF6α was mainly observed outside and around the nucleus. Following TM treatment, while the cleaved ATF6α p50 was translocated to the nucleus in the control cells; in siBBS4 cells it failed to translocate and remained in the ER. The failure of the cleaved ATF6α p50 to translocate to the nucleus is clearly indicated in the quantification of cleaved ATF6α p50 in the nucleus (FIG. 9B).

In the next step, the level of expression of ATF6α was determined. Under UPR activation (TM treatment), both siBBS4 and control SH-SY5Y cells significantly (P<0.01) and similarly up-regulated ATF6α transcripts (1.3-fold and 1.5-fold, respectively) and full-length protein (2.4-fold and 2.3-fold, respectively) levels (FIG. 9D). However, significant reduction in the cleaved active ATF6α p50 form was observed in siBBS4 compared to the control SH-SY5Y cells and to the basal/normal state (FIG. 9D) in both untreated and TM—treated states. ATF6α transcript levels did not differ between siBBS4 and control cells under normal or stress conditions (FIG. 9C); suggestively indicating that BBS4 is not involved in ATF6α transcript regulation, but rather in the post-transcriptional regulation at the protein level and to the trans-localization to the nucleus.

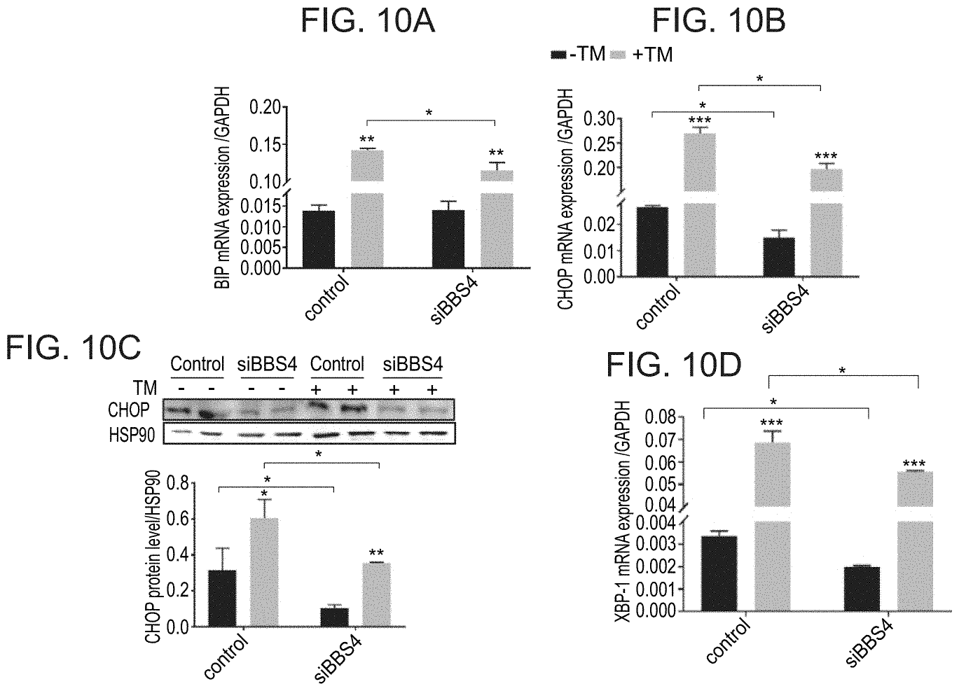

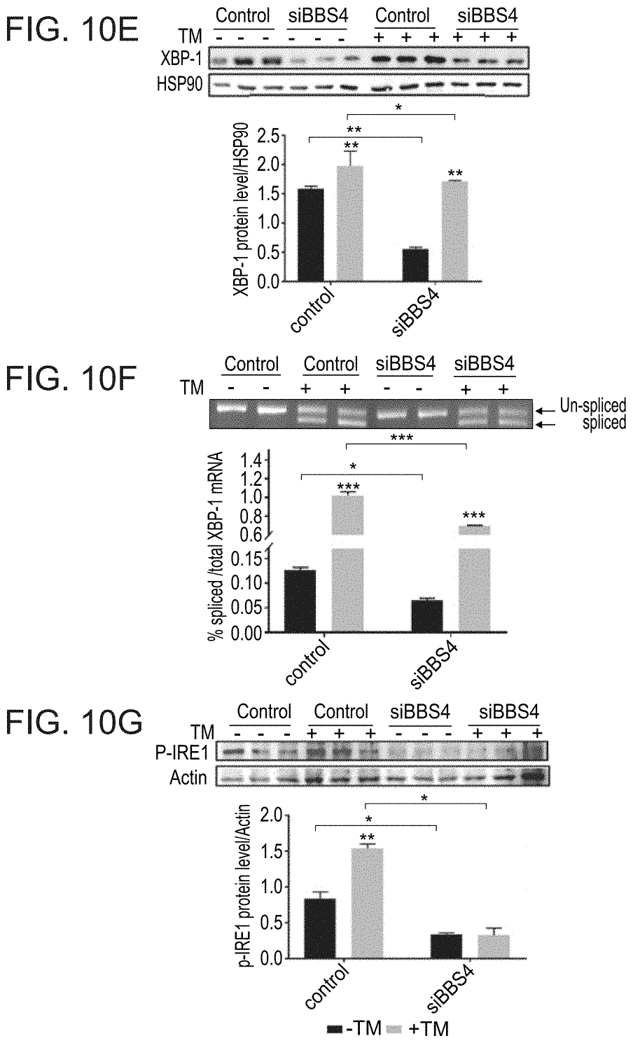

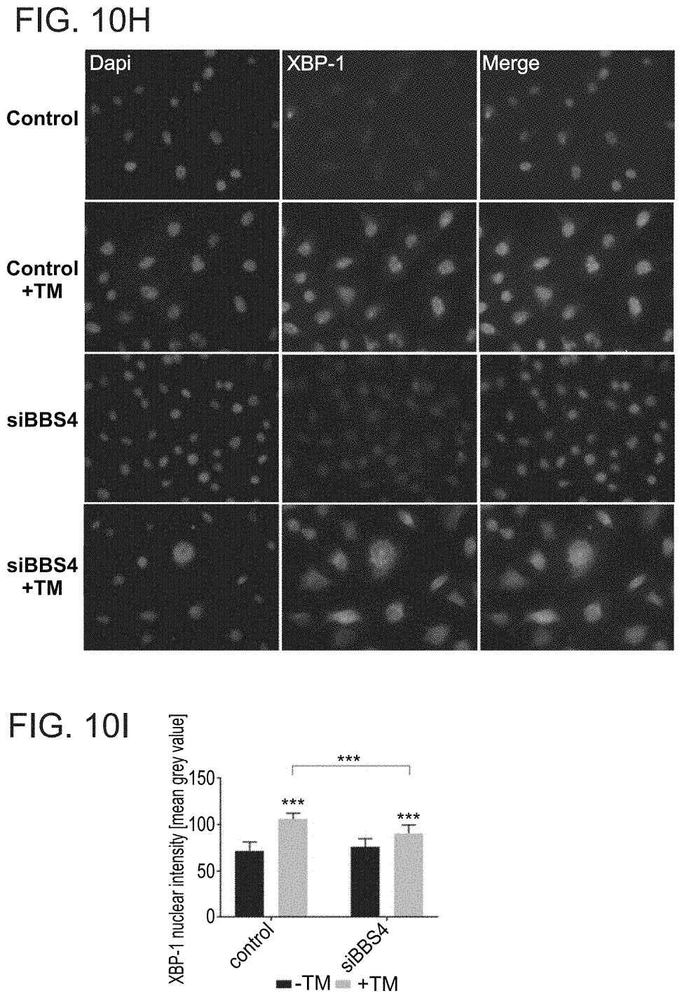

Under ER stress conditions the molecular chaperone BiP dissociates from the ER stress sensors (e.g. ATF6α). Under non-stressed conditions, BiP transcript levels did not differ significantly between siBBS4 and control SH-SY5Y cells. Following TM treatment, although BiP transcript levels were significantly (P>0.001) up-regulated in control and siBBS4 cells, siBBS4 SH-SY5Y demonstrated significantly reduced Bip levels (FIG. 10A).

Further, TM-induced ER stress resulted in significant (P<0.001) up-regulation of CHOP protein and transcript levels in both siBBS4 and control SH-SY5Y cells. Specifically, in control cells, TM treatment elevated CHOP levels by 26-fold and 3-fold (transcript and protein, respectively), and in siBBS4 cells by 26-fold and 3.4-fold (transcript and protein, respectively) compared to untreated cells (control and siBSB4, respectively). Yet, TM-induced up-regulation of CHOP levels were significantly (P<0.05) reduced in siBBS4 cells by 1.6 (transcript) and 1.7 (protein) fold compared to control cells (FIG. 10B-C).

Similarly, although SiBBS4 cells showed a significant (P<0.01) elevation in XBP-1 transcript and protein levels following TM-induced ER stress, the levels were significantly (P<0.05) lower by 2.8 (protein) and 1.2 (transcript)-fold compared to the levels in control treated cells (FIG. 10D-E). Moreover, upon TM-induced ER stress, the percentages of sXBP1 levels were significantly up regulated (P<0.001) in both siBBS4 and control SH-SY5Y cells. However, the percentages of sXBP1 levels in siBBS4 were significantly (P<0.01) reduced by 1.5-fold compared to TM-treated control SH-SY5Y cells (FIG. 10F). In the next step, the levels of pIRE1α, which splices the XBP-1 mRNA, were analyzed. As expected, in control cells, TM induction resulted in significant (P<0.05) upregulation of pIRE-1α by ˜2-fold compared to untreated control cells. Interestingly, untreated and TM-treated siBBS4 cells demonstrated similar and significantly (P<0.05) reduced pIRE-1α levels by 2.5-fold and 4.7-fold, respectively, compared to control cells (FIG. 10G). Upon UPR induction, sXBP-1 is transported to the nucleus and acts as a transcription factor. In order to investigate whether BBS4 knock down influences XBP-1 transport and subcellular localization, XBP-1 location following TM treatment was studied using immunohistochemistry. In untreated control and siBBS4 SH-SY5Y cells, XBP-1 was located to the cytoplasm. As expected, following TM-induced ER stress, sXBP-1 was translocated to the nucleus in the control SH-SY5Y cells; however, in siBBS4 SH-SY5Y cells sXBP-1 failed to translocate to the nucleus as indicated by accumulation of sXBP-1 in the cytoplasm and ER, suggesting abrogated sXBP-1 transport to the nucleus (FIG. 10H-I).

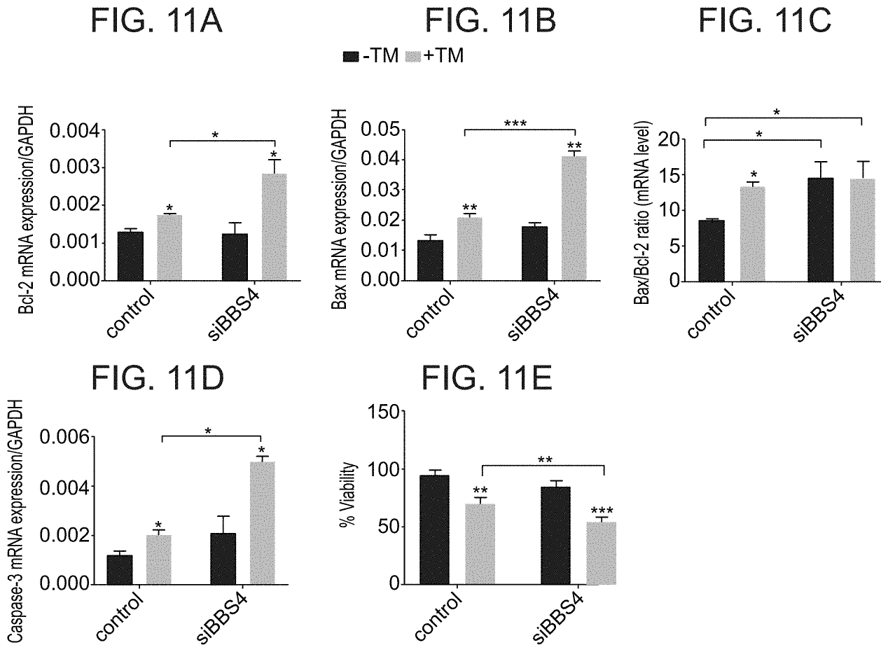

BBS4 silencing elevates apoptosis markers under TM-induced ER stress—Sustained ER stress results in prolonged activation of the UPR and disability to regain homeostasis and is associated with the initiation of apoptotic pathways. To this end, the transcripts expression of the apoptotic markers B-cell lymphoma 2 (Bcl-2) (anti-apoptosis marker), Bcl-2 associated X-protein (Bax) (pro-apoptosis marker) and caspase-3 were studied in TM-treated siBBS4 and control SH-SY5Y cells. Following ER stress induction, Bcl-2 transcript levels were significantly (P<0.05) up-regulated by 2.3-fold and 1.3-fold in siBBS4 and control cells, respectively. TM-treated siBBS4 cells exhibited a significantly (P<0.05) higher elevation in Bcl-2 levels, by 1.6-fold compared to TM-treated control cells (FIG. 11A). Similarly, ER stress induction significantly (P<0.0.5) increased Bax transcript levels by 2.3-fold and 1.5-fold in siBSB4 and control SH-SY5Y cells, respectively. However, TM-treated siBBS4 cells elevated Bax transcript to significant (P<0.01) higher levels, 2-fold compared to TM-treated control cells (FIG. 11B). The Bax/Bcl-2 ratio is used as a determining factor for the induction of apoptosis. As expected, TM treatment significantly (P<0.05) up-regulated Bax/BCl-2 ratio in control SH-SY5Y cells by 1.5-fold. SiBBS4 cells exhibited a significantly (P<0.05) higher Bax/Bcl-2 ratio, 1.7-fold compared to non-stressed control cells, regardless the ER stress level (FIG. 11C). Correspondingly, Caspase-3 transcript levels were significantly (P<0.05) up-regulated in siBBS4 and control cells in response to ER stress induction, by 2.4-fold and 1.3-fold compared to untreated cells, respectively. Yet, siBBS4 cells showed a significant (P<0.05) elevation in caspase-3 up-regulation following TM treatment, 2.5-fold compared to TM-treated control cells (FIG. 11D). Next, the viability level in siBBS4 and control cells 24 hours following TM treatment urs was measured. In response to ER stress induction, % viability was significantly (P<0.01) down-regulated by 1.4-fold and 1.6-fold in both control and siBBS4, respectively. However, TM-treated siBBS4 cells exhibited a significant (P<0.01) reduction of 1.3-fold in % viability compared to TM-treated control cells (FIG. 11E). These results indicate an intensified activation of apoptosis pathways under BBS4 depletion.

### Example 4

### Down-Regulation of BBS4 Affects Proliferation and Differentiation of Neuronal Cells

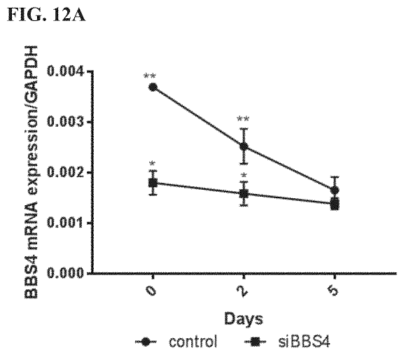

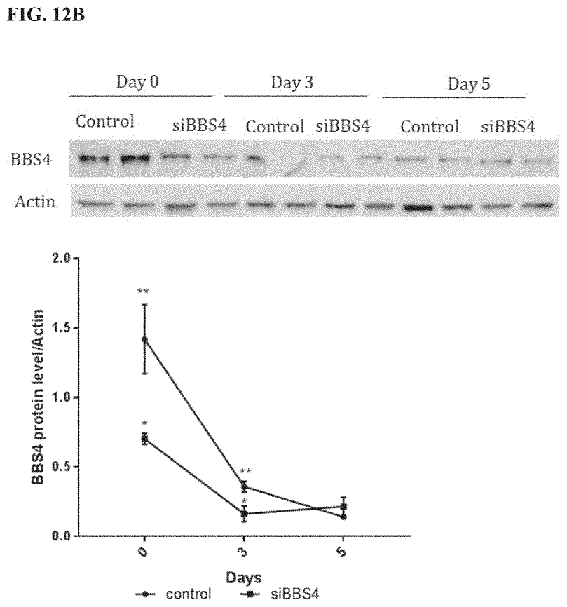

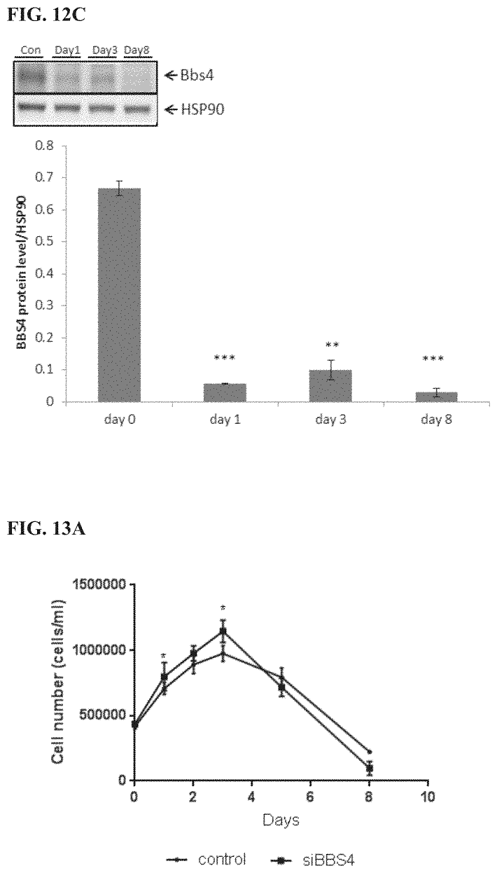

BBS4 expression is downregulated during neural differentiation—Neural differentiation is the process by which premature neural cells differentiate into mature neuron cells. To study the role of BBS4 in neural differentiation, BBS4 transcript and protein levels were analyzed during neuronal differentiation of SH-SY5Y and PC-12 cells. As shown in FIG. 12A-B, in SH-SY5Y cells BBS4 mRNA and protein levels were significantly (P<0.05) decreased during differentiation, reaching the lowest expression at day 5. Similarly, in PC-12 cells, BBS4 protein levels were significantly (P<0.05) decreased during differentiation, reaching the lowest expression after 8 days of differentiation (FIG. 12C). siBBS4 significantly reduced BBS4 levels in both SH-SY5Y and PC-12 cells as compared to the control cells, reflecting a valid and reliable knock down model.

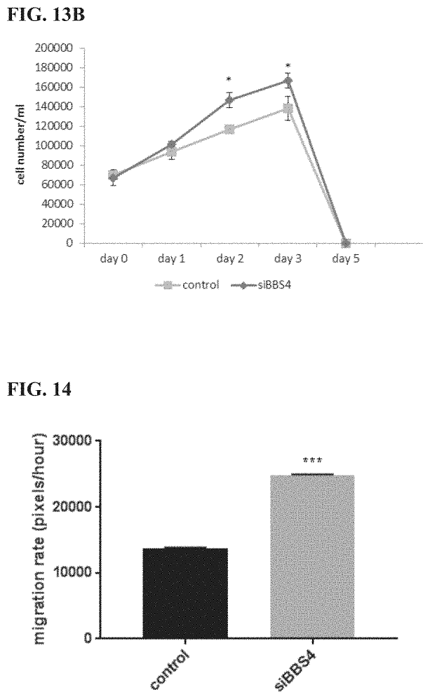

BBS4 silencing increases neuronal proliferation—Neural differentiation and maturation are accompanied with cell proliferation arrest. To this end, cell proliferation rate was analyzed in siBBS4 and control SH-SY5Y and PC-12 cells throughout differentiation, starting at day 0 (undifferentiated cells) and follow by replacement of culture medium to differentiation medium for 8 days (SH-SY5Y cells) or 10 days (PC-12 cells). As seen in FIG. 13A, SH-SY5Y siBBS4 cells proliferated more rapidly compared to SH-SY5Y control cells and reached a significant cell number difference at days 1, 3 of differentiation. By day 5 of differentiation, no significant difference was found in proliferation. Notably, both siBBS4 and control PC-12 cells survive in differentiation medium for 3 days of differentiation and died past these days. However, siBBS4 PC-12 cell number measured in days 2-3 of differentiation was significantly (P<0.05) higher compared to control PC-12 cells (FIG. 13B).

BBS4 silencing increases neuronal migration—In order to demonstrate BBS4 role in migration, a wound healing assay was performed for migration rate quantification in siBBS4 and control SH-SY5Y cells. As shown in FIG. 14, siBBS4 cells migration rate was significantly (P<0.001) higher by ˜2 fold compared to control cells, reflecting a higher proliferation rate and consequently increased wound healing and regeneration abilities under BBS4 knock-down conditions.

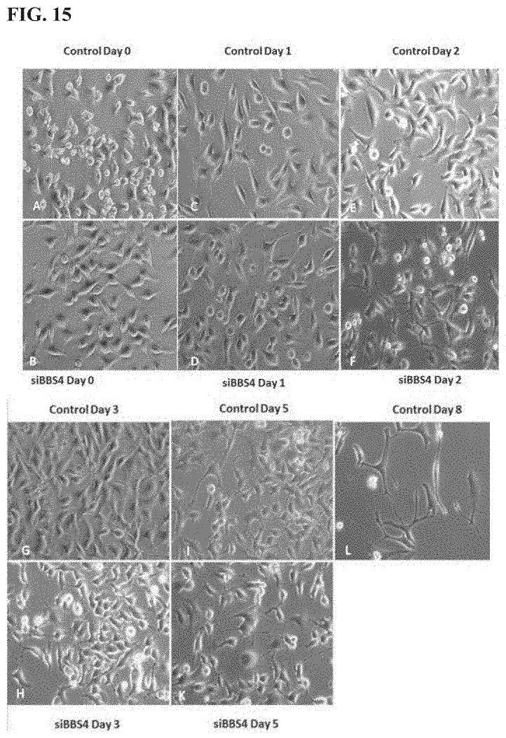

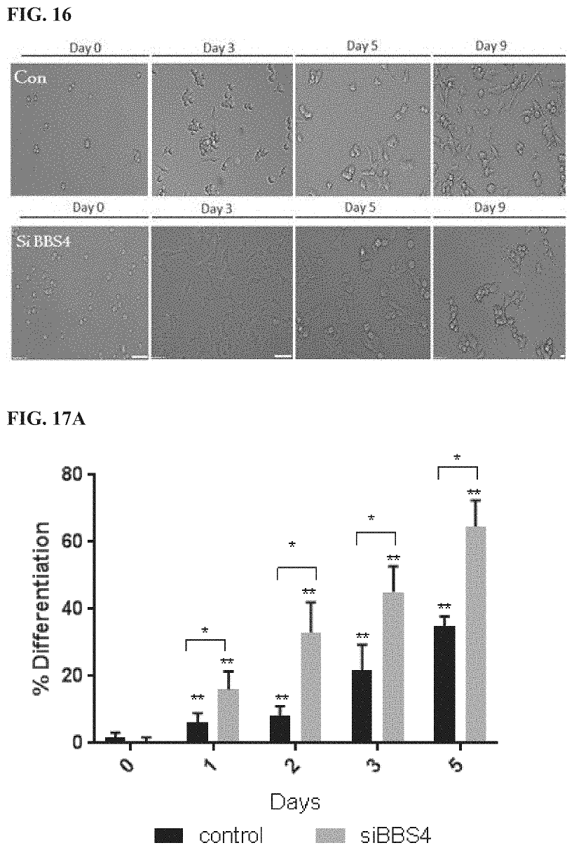

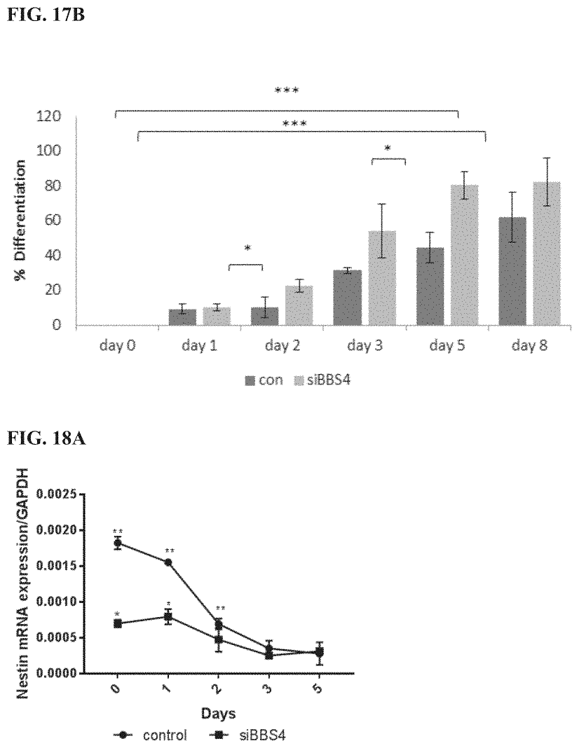

BBS4 silencing affects cells' morphology—Neural differentiation induces morphological changes characteristic of neurons, for example extension of neurites. To this end, neurite outgrowths in siBBS4 and control SH-SY5Y and PC-12 cells during differentiation was studied using. Briefly, cells bearing at least one neurite with the length equivalent to cell bodies were scored as differentiated cells. For SH-SY5Y cells differentiation, siBBS4 and control cells were cultured in a differentiation medium supplemented with 10 μM retinoic acid (RA) for 8 days, and morphologically was microscopically assessed at days 0, 1, 2, 3, 5 of differentiation. For PC-12 cells differentiation, siBBS4 and control cells were cultured in a neuron differentiation medium supplemented with 50 ng/μl β-nerve growth factor (0-NGF) for 11 days, and morphologically was microscopically assessed at days 0, 3, 9, 11 of differentiation. At day 0 of differentiation, both siBBS4 and control cells in both cell lines were undifferentiated, had no neurites and showed no significant morphological differences. As seen in FIGS. 15, 16 and 17A-B, culturing of both cell lines in differentiation media induced neural differentiation in both siBBS4 and control cells, reflected by continual neurite outgrowths along differentiation days. However, siBBS4 cells showed significant accelerated neurites growth compared to control cells in both cell lines. Specifically, in SH-SY5Y cells already following one day of differentiation, siBBS4 cells already exhibited more neurites compared to control cells, a tendency maintained throughout the differentiation process (days 2-5 of differentiation). During days 2, 3, 5 of differentiation, more differentiated siBBS4 cells were scored positive in comparison to the control cells (FIG. 17A), reaching a peak of about 65% of cells at day 5 of differentiation compared to 30% of control cells. As control SH-SY5Y cells survived in differentiation medium for 8 days and siBBS4 cells survived for 5 days in the same medium (FIGS. 15 and 17A), no further analysis was effected in this model past day 5 of differentiation. Similarly, in PC-12 cells following two days of NGF treatment siBBS4 cells already exhibited more neurites compared to control cells, a tendency maintained throughout early differentiation (days 0-5 of differentiation) (FIGS. 16 and 17B). Taken together, different differentiation rate in siBBS4 cells compared with control cells suggest the involvement of BBS4 in the differentiation process of SH-SY5Y and PC-12 neuronal cell line.

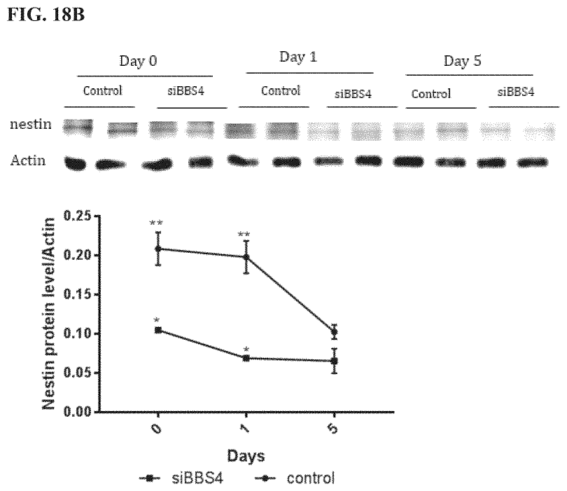

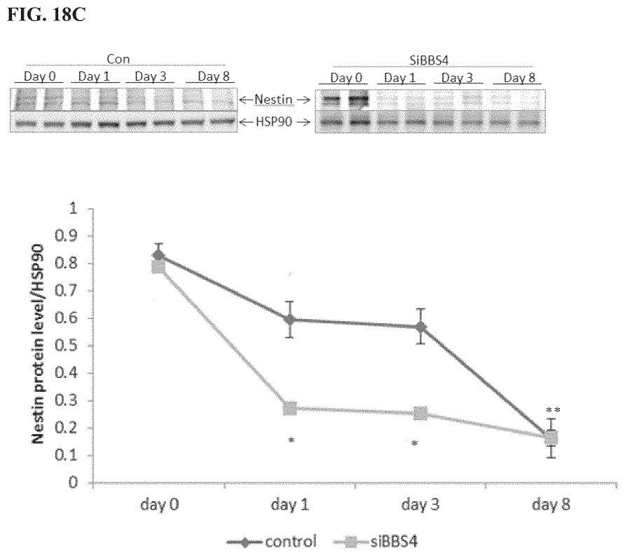

BBS4 silencing accelerates neural differentiation—Differentiation of neuronal cell is characterized by a gene expression switch. Undifferentiated SH-SY5Y and PC-12 pre-neuron expresses immature neuronal markers, whereas differentiated cells exit the cell cycle and show increased expression of a variety of neuron specific markers. Nestin, an intermediate filament protein that promotes the activation of PI3K pathway, is considered being the most common early marker for neural differentiation. Specifically, nestin is expressed mostly in dividing premature neurons; and upon differentiation nestin expression is downregulated, reaching a low expression level at late differentiation stage. To this end, nestin expression was analyzed during SH-SY5Y and PC-12 cells differentiation. FIGS. 18A-B show nestin levels in SH-SY5Y during 5 days of differentiation. As shown, both nestin transcript and protein levels significantly (P<0.01) decreased during differentiation in SH-SY5Y control cells, indicating a proper neuronal cell maturation and differentiation. For example, nestin transcript expression levels in control cells were significantly (P<0.01) higher by 6.5 fold in day 0 compared to day 5. A similar decrease in nestin transcript levels was observed in siBBS4 cells, with a significant (P<0.05) reduction of 2.2 fold in day5 compared to day 0. However, in the siBBS4 cells, nestin transcript levels were significantly (P<0.05) lower compared to the control cells throughout early differentiation days (days 0-1): For example, at day 0, nestin transcript levels in siBBS4 cells were significantly (P<0.01) lower by 2.6 fold compared to control; likewise, at day 1 nestin expression in siBBS4 was significantly (P<0.05) lower by 1.9 cells in comparison to control cells. Notably, nestin levels in siBBS4 cells differed significantly (P<0.05) from the control cells until day 2 of differentiation, while as differentiation progressed nestin expression reached a similar and lower levels in both siBBS4 and control, and no significant difference was observed following 5 day of differentiation (FIGS. 18A-B). In a similar manner, in the PC-12 cell line, nestin protein levels significantly (P<0.01) decreased during differentiation in control cells, indicating a proper neuronal cell maturation and differentiation in this cell line as well (FIG. 18C). Specifically, nestin protein levels in the control cells were significantly (P<0.01) higher by 5.1-fold in day 0 compared to day 8. A similar decrease in nestin protein levels was observed in the siBBS4 cells, with a significant (P<0.05) reduction by 4.8-fold in day 5 compared to day 0. While in un-differentiated PC-12 cells, nestin protein levels did not significantly differ between control and siBBS4 cells; during early differentiation (days 1-3) nestin protein levels were significantly (P<0.05) lower in the siBBS4 cells compared to the control cells. For example, at days 1 and 3, nestin protein levels in siBBS4 cells were significantly (P<0.05) downregulated by 2.2-fold in comparison to control cells. At later differentiation days (day 8), nestin protein levels in both siBBS4 and control cells reached similar low levels, and no significant difference was observed by day 8 of differentiation. Taken together, these results further indicate an involvement of BBS4 in the maturation of SH-SY5Y and PC-12 neuronal cell lines.

Taken together, BBS4 protein and transcript levels are down-regulated during neuronal differentiation. In undifferentiated state, BBS4 silencing resulted in significantly reduced ER stress markers expression (namely CHOP, XBP-1, cleaved ATF6, spliced XBP-1, BIP, pIRE1α) and reduced translocation of sXBP-1 and the activated cleaved ATF6 to the nucleus, under both non-stressed and TM-induced ER stress states. Furthermore, BBS4 silencing and ER stress induction resulted in significant upregulation of transcript levels of apoptosis markers (Bax, Bcl-2, Caspase-3), corresponding to decreased viability. In addition BBS4 silencing increased differentiation, proliferation and migration of neuronal cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1. Andrade, L. J. D. O., Andrade, R., Franga, C. S., & Bittencourt, A. V. (2009). Pigmentary retinopathy due to Bardet-Biedl syndrome: case report and literature review. Arquivos brasileiros de oftalmologia, 72(5), 694-696.
2. Priya, S., Nampoothiri, S., Sen, P., & Sripriya, S. (2016). Bardet-Biedl syndrome: Genetics, molecular pathophysiology, and disease management. Indian journal of ophthalmology, 64(9), 620.
3. Forsythe, E., & Beales, P. L. (2013). Bardet-Biedl syndrome. European journal of human genetics, 21(1), 8.
4. Redin, C., Le Gras, S., Mhamdi, O., Geoffroy, V., Stoetzel, C., Vincent, M. C., & Till, M. (2012). Targeted high-throughput sequencing for diagnosis of genetically heterogeneous diseases: efficient mutation detection in Bardet-Biedl and Alström syndromes. Journal of medical genetics, 49(8), 502-512.
5. Hjortshøj, T. D., Grønskov, K., Brøndum-Nielsen, K., & Rosenberg, T. (2009). A novel founder BBS1 mutation explains a unique high prevalence of Bardet-Biedl syndrome in the Faroe Islands. British Journal of Ophthalmology, 93(3), 409-413.
6. Fliegauf, M., Benzing, T., & Omran, H. (2007). When cilia go bad: cilia defects and ciliopathies. Nature reviews Molecular cell biology, 8(11), 880.
7. Nachury, M. V., Loktev, A. V., Zhang, Q., Westlake, C. J., Peränen, J., Merdes, A., . . . & Jackson, P. K. (2007). A core complex of BBS proteins cooperates with the GTPase Rab8 to promote ciliary membrane biogenesis. Cell, 129(6), 1201-1213.
8. Álvarez-Satta, M., Castro-Sánchez, S., & Valverde, D. (2017). Bardet-Biedl Syndrome as a Chaperonopathy: Dissecting the Major Role of Chaperonin-Like BBS Proteins (BBS6-BBS10-BBS12). Frontiers in molecular biosciences, 4, 55.
9. Moss, J., & Vaughan, M. (1995). Structure and function of ARF proteins: activators of cholera toxin and critical components of intracellular vesicular transport processes. Journal of Biological Chemistry, 270(21), 12327-12330.
10. Gregor, M. F., & Hotamisligil, G. S. (2007). Thematic review series: Adipocyte Biology. Adipocyte stress: the endoplasmic reticulum and metabolic disease. Journal of lipid research, 48(9), 1905-1914.
11. Chiang, A. P., Beck, J. S., Yen, H. J., Tayeh, M. K., Scheetz, T. E., Swiderski, R. E., & Elbedour, K. (2006). Homozygosity mapping with SNP arrays identifies TRIM32, an E3 ubiquitin ligase, as a Bardet-Biedl syndrome gene (BBS11). Proceedings of the National Academy of Sciences, 103(16), 6287-6292.
12. Xu, Q., Zhang, Y., Wei, Q., Huang, Y., Li, Y., Ling, K., & Hu, J. (2015). BBS4 and BBS5 show functional redundancy in the BBSome to regulate the degradative sorting of ciliary sensory receptors. Scientific reports, 5, 11855.
13. Chang, B., Khanna, H., Hawes, N., Jimeno, D., He, S., Lillo, C., & Sayer, J. A. (2006). In-frame deletion in a novel centrosomal/ciliary protein CEP290/NPHP6 perturbs its interaction with RPGR and results in early-onset retinal degeneration in the rd16 mouse. Human molecular genetics, 15(11), 1847-1857.
14. Seo, S., Guo, D. F., Bugge, K., Morgan, D. A., Rahmouni, K., & Sheffield, V. C. (2009). Requirement of Bardet-Biedl syndrome proteins for leptin receptor signaling. Human molecular genetics, 18(7), 1323-1331.
15. Rahmouni, K., Fath, M. A., Seo, S., Thedens, D. R., Berry, C. J., Weiss, R., & Sheffield, V. C. (2008). Leptin resistance contributes to obesity and hypertension in mouse models of Bardet-Biedl syndrome. The Journal of clinical investigation, 118(4), 1458-1467.
16. Seo, S., Guo, D. F., Bugge, K., Morgan, D. A., Rahmouni, K., & Sheffield, V. C. (2009). Requirement of Bardet-Biedl syndrome proteins for leptin receptor signaling. Human molecular genetics, 18(7), 1323-1331.
17. Forti, E., Aksanov, O., & Birk, R. Z. (2007). Temporal expression pattern of Bardet-Biedl syndrome genes in adipogenesis. The international journal of biochemistry & cell biology, 39(5), 1055-1062.
18. Nahum, N., Forti, E., Aksanov, O., & Birk, R. (2017). Insulin regulates Bbs4 during adipogenesis. IUBMB life, 69(7), 489-499.
19. Aksanov, O., Green, P., & Birk, R. Z. (2014). BBS4 directly affects proliferation and differentiation of adipocytes. Cellular and molecular life sciences, 71(17), 3381-3392.
20. Marion, V., Stoetzel, C., Schlicht, D., Messaddeq, N., Koch, M., Flori, E., & Dollfus, H. (2009). Transient ciliogenesis involving Bardet-Biedl syndrome proteins is a fundamental characteristic of adipogenic differentiation. Proceedings of the National Academy of Sciences, 106(6), 1820-1825.
21. Yilmaz, E. (2017). Endoplasmic reticulum stress and obesity. In Obesity and Lipotoxicity (pp. 261-276). Springer, Cham.
22. Gregor, M. F., & Hotamisligil, G. S. (2007). Thematic review series: Adipocyte Biology. Adipocyte stress: the endoplasmic reticulum and metabolic disease. Journal of lipid research, 48(9), 1905-1914.
23. Gregor, M. F., Yang, L., Fabbrini, E., Mohammed, B. S., Eagon, J. C., Hotamisligil, G S., & Klein, S. (2009). Endoplasmic reticulum stress is reduced in tissues of obese subjects after weight loss. Diabetes, 58(3), 693-700.
24. Kawasaki, N., Asada, R., Saito, A., Kanemoto, S., & Imaizumi, K. (2012). Obesity-induced endoplasmic reticulum stress causes chronic inflammation in adipose tissue. Scientific reports, 2, 799.
25. Ariyasu, D., Yoshida, H., & Hasegawa, Y. (2017). Endoplasmic reticulum (ER) stress and endocrine disorders. International journal of molecular sciences, 18(2), 382.
26. Shen, J., Chen, X., Hendershot, L., & Prywes, R. (2002). ER stress regulation of ATF6 localization by dissociation of BiP/GRP78 binding and unmasking of Golgi localization signals. Developmental cell, 3(1), 99-111.
27. Ye, J., Rawson, R. B., Komuro, R., Chen, X., Davé, U. P., Prywes, R., & Goldstein, J. L. (2000). ER stress induces cleavage of membrane-bound ATF6 by the same proteases that process SREBPs. Molecular cell, 6(6), 1355-1364.
28. Gascue, C., Tan, P. L., Cardenas-Rodriguez, M., Libisch, G, Fernandez-Calero, T., Liu, Y. P., & Badano, J. L. (2012). Direct role of Bardet-Biedl syndrome proteins in transcriptional regulation. J Cell Sci, 125(2), 362-375.
29. Negi, S., Pandey, S., Srinivasan, S. M., Mohammed, A., & Guda, C. (2015). LocSigDB: a database of protein localization signals. Database, 2015.
30. Jung, U. J., & Choi, M. S. (2014). Obesity and its metabolic complications: the role of adipokines and the relationship between obesity, inflammation, insulin resistance, dyslipidemia and nonalcoholic fatty liver disease. International journal of molecular sciences, 15(4), 6184-6223.
31. Emanuela, F., Grazia, M., Marco, D. R., Maria Paola, L., Giorgio, F., & Marco, B. (2012). Inflammation as a link between obesity and metabolic syndrome. Journal of nutrition and metabolism, 2012.
32. Balistreri, C. R., Caruso, C., & Candore, G (2010). The role of adipose tissue and adipokines in obesity-related inflammatory diseases. Mediators of inflammation, 2010.
33. Mockel, A., Obringer, C., Hakvoort, T. B., Seeliger, M., Lamers, W. H., Stoetzel, C., & Marion, V. (2012). Pharmacological modulation of the retinal unfolded protein response in Bardet-Biedl syndrome reduces apoptosis and preserves light detection ability. Journal of Biological Chemistry, 287(44), 37483-37494.
34. Swiderski, R. E., Nishimura, D. Y., Mullins, R. F., Olvera, M. A., Ross, J. L., Huang, J.,& Sheffield, V. C. (2007). Gene expression analysis of photoreceptor cell loss in bbs4-knockout mice reveals an early stress gene response and photoreceptor cell damage.

Investigative ophthalmology & visual science, 48(7), 3329-3340.
35. Longo, M., Spinelli, R., D'Esposito, V., Zatterale, F., Fiory, F., Nigro, C., & Di Jeso, B. (2016). Pathologic endoplasmic reticulum stress induced by glucotoxic insults inhibits adipocyte differentiation and induces an inflammatory phenotype. Biochimica et Biophysica Acta (BBA)-Molecular Cell Research, 1863(6), 1146-1156.
36. Oslowski, C. M., & Urano, F. (2011). The binary switch that controls the life and death decisions of ER stressed 0 cells. Current opinion in cell biology, 23(2), 207-215.
37. Sha, H., He, Y., Chen, H., Wang, C., Zenno, A., Shi, H., & Qi, L. (2009). The IRE1α-XBP1 pathway of the unfolded protein response is required for adipogenesis. Cell metabolism, 9(6), 556-564.
38. Han, J., Murthy, R., Wood, B., Song, B., Wang, S., Sun, B., & Kaufman, R. J. (2013). ER stress signalling through eIF2a and CHOP, but not IRE1α, attenuates adipogenesis in mice. Diabetologia, 56(4), 911-924.
39. Lowe, C. E., Dennis, R. J., Obi, U., O'rahilly, S., & Rochford, J. J. (2012). Investigating the involvement of the ATF6α pathway of the unfolded protein response in adipogenesis. International journal of obesity, 36(9), 1248.
40. Rangwala, S. M., & Lazar, M. A. (2000). Transcriptional control of adipogenesis.

Annual review of nutrition, 20(1), 535-559.
41. Prieto-Echagie, V., Lodh, S., Colman, L., Bobba, N., Santos, L., Katsanis, N., & Badano, J. L. (2017). BBS4 regulates the expression and secretion of FSTL1, a protein that participates in ciliogenesis and the differentiation of 3T3-L1. Scientific Reports, 7(1), 9765.
42. Lechtreck, K. F., Brown, J. M., Sampaio, J. L., Craft, J. M., Shevchenko, A., Evans, J. E., & Witman, G B. (2013). Cycling of the signaling protein phospholipase D through cilia requires the BBSome only for the export phase. J Cell Biol, 201(2), 249-261.
43. Wei, Q., Zhang, Y., Li, Y., Zhang, Q., Ling, K., & Hu, J. (2012). The BBSome controls IFT assembly and turnaround in cilia. Nature cell biology, 14(9), 950.
44. Jin, H., White, S. R., Shida, T., Schulz, S., Aguiar, M., Gygi, S. P., & Nachury, M. V. (2010). The conserved Bardet-Biedl syndrome proteins assemble a coat that traffics membrane proteins to cilia. Cell, 141(7), 1208-1219.
45. Tinahones, F. J., Aragüez, L. C., Murri, M., Olivera, W. O., Torres, M. D. M., Barbarroja, N., & El Bekay, R. (2013). Caspase induction and BCL2 inhibition in human adipose tissue: a potential relationship with insulin signaling alteration. Diabetes care, 36(3), 513-521.
46. Salakou, S., Kardamakis, D., Tsamandas, A. C., Zolota, V., Apostolakis, E., Tzelepi, V., & Dougenis, D. (2007). Increased Bax/Bcl-2 ratio up-regulates caspase-3 and increases apoptosis in the thymus of patients with myasthenia gravis. In vivo, 21(1), 123-132.
47. Xiong, Y., Chen, H., Lin, P., Wang, A., Wang, L., & Jin, Y. (2017). ATF6 knockdown decreases apoptosis, arrests the S phase of the cell cycle, and increases steroid hormone production in mouse granulosa cells. American Journal of Physiology-Cell Physiology, 312(3), C341-C353.
48. Hargitai, B., Szabo, V., Hajdu, J., Harmath, A., Pataki, M., Farid, P., . . . & Szende, B. (2001). Apoptosis in various organs of preterm infants: histopathologic study of lung, kidney, liver, and brain of ventilated infants. Pediatric research, 50(1), 110.
49. Brill, A., Torchinsky, A., Carp, H., & Toder, V. (1999). The role of apoptosis in normal and abnormal embryonic development. Journal of assisted reproduction and genetics, 16(10), 512-519.
50. Kam, P. C. A., & Ferch, N. I. (2000). Apoptosis: mechanisms and clinical implications. Anaesthesia, 55(11), 1081-1093.
51. ISO 690 Herold, C., Rennekampff, H. O., & Engeli, S. (2013). Apoptotic pathways in adipose tissue. Apoptosis, 18(8), 911-916.
52. Tung, C. H., Han, M. S., & Qi, J. (2017). Total control of fat cells from adipogenesis to apoptosis using a xanthene analog. PloS one, 12(6), e0179158.
53. Saveljeva, S., Mc Laughlin, S. L., Vandenabeele, P., Samali, A., & Bertrand, M. J. (2015). Endoplasmic reticulum stress induces ligand-independent TNFR1-mediated necroptosis in L929 cells. Cell death & disease, 6:e1587.
54. Fan, H., Tang, H. B., Kang, J., Shan, L., Song, H., Zhu, K., Wang, J., Ju, G, & Wang, Y. Z. (2015). Involvement of endoplasmic reticulum stress in the necroptosis of microglia/macrophages after spinal cord injury. Neuroscience, 311:362-373.
55. Parlee, S. D., Lentz, S. I., Mori, H., & MacDougald, O. A. (2014). Quantifying size and number of adipocytes in adipose tissue. In Methods in enzymology (Vol. 537, pp. 93-122). Academic Press.

1. A method of treating a disease associated with cells exhibiting ER stress in a subject in need thereof, the method comprising administering to the subject a therapeutically effective amount of an agent which downregulates expression and/or activity of BBS, thereby treating the disease associated with cells exhibiting ER stress in the subject.

2. (canceled)

3. The method of claim 1, wherein said disease is selected from the group consisting of cancer, an inflammatory disease, a metabolic disease and infection.

4-5. (canceled)

6. A method of forming or regenerating a neural tissue, the method comprising contacting neuronal stem or progenitor cells with an agent which downregulates expression and/or activity of BBS, thereby forming or regenerating the neural tissue.

7. A method of treating a subject having a disease that can benefit from neural tissue formation or regeneration, the method comprising administering to the subject a therapeutically effective amount of an agent which downregulates expression and/or activity of BBS, thereby treating the disease that can benefit from neural tissue formation or regeneration in the subject.

8. (canceled)

9. The method of claim 7, wherein said disease is selected from the group consisting of neurodegenerative disease, ischemia, stroke, neuronal loss associated with aging and nerve injury caused by trauma.

10. The method of claim 6, wherein said contacting is effected in-vitro or ex-vivo.

11. The method of claim 6, wherein said contacting is effected in-vivo.

12. The method of claim 1, wherein said agent is an RNA silencing agent.

13. The method of claim 1, wherein said agent is an aptamer, a peptide or a small molecule.

14. (canceled)

15. The method of claim 1, wherein said BBS is not BBS12.

16. The method of claim 1, wherein said BBS is selected from the group consisting of BBS1, BBS2, BBS3, BBS4, BBS5, BBS6, BBS7, BBS8, BBS9, BBS10, BBS11, BBS12, BBS13, BBS14, BBS15, BBS16, BBS17, BBS18, BBS19, BBS20 and BBS21.

17. The method of claim 1, wherein said BBS is selected from the group consisting of BBS1, BBS2, BBS3, BBS4, BBS5, BBS6, BBS7, BBS8, BBS9, BBS10, BBS11, BBS13, BBS14, BBS15, BBS16, BBS17, BBS18, BBS19, BBS20 and BBS21.

18. The method claim 1, wherein said BBS is selected from the group consisting of BBS1, BBS2, BBS4, BBS5, BBS7, BBS8, BBS9 and BBS18.

19. The method of claim 1, wherein said BBS comprises BBS4.

20. The method of claim 1, wherein downregulating activity of said BBS comprises affecting localization of said BBS.


SEQUENCE LISTING (50 sequences)

SEQ ID NO: 1 - DNA, 20 bp, Artificial sequence
  Single strand DNA oligonucleotide
ccataacctgggagtgtgct

SEQ ID NO: 2 - DNA, 20 bp, Artificial sequence
  Single strand DNA oligonucleotide
tcgatggctttatccaggtc

SEQ ID NO: 3 - DNA, 20 bp, Artificial sequence
  Single strand DNA oligonucleotide
attctgacgctgttgcctct

SEQ ID NO: 4 - DNA, 20 bp, Artificial sequence
  Single strand DNA oligonucleotide
aaagggaggctggtaaggaa

SEQ ID NO: 5 - DNA, 22 bp, Artificial sequence
  Single strand DNA oligonucleotide
ttacgagagaaaactcatgggc

SEQ ID NO: 6 - DNA, 22 bp, Artificial sequence
  Single strand DNA oligonucleotide
gggtccaacttgtccagaatgc

SEQ ID NO: 7 - DNA, 21 bp, Artificial sequence
  Single strand DNA oligonucleotide
ctggaagcctggtatgaggat

SEQ ID NO: 8 - DNA, 23 bp, Artificial sequence
  Single strand DNA oligonucleotide
gcagggtcaagagtagtgaaggt

SEQ ID NO: 9 - DNA, 20 bp, Artificial sequence
  Single strand DNA oligonucleotide
ggccagactgttttgctctc

SEQ ID NO: 10 - DNA, 20 bp, Artificial sequence
  Single strand DNA oligonucleotide
cccatacttctggtggcact

SEQ ID NO: 11 - DNA, 19 bp, Artificial sequence
  Single strand DNA oligonucleotide
ctgggatgcctttgtggaa

SEQ ID NO: 12 - DNA, 20 bp, Artificial sequence
  Single strand DNA oligonucleotide
tcaaacagaggtcgcatgct

SEQ ID NO: 13 - DNA, 20 bp, Artificial sequence
  Single strand DNA oligonucleotide
agcttggaacggtacgctaa

SEQ ID NO: 14 - DNA, 20 bp, Artificial sequence
  Single strand DNA oligonucleotide
cgtaccagagcgagatgaca

SEQ ID NO: 15 - DNA, 19 bp, Artificial sequence
  Single strand DNA oligonucleotide
agtgtctccggcgaattgg

SEQ ID NO: 16 - DNA, 21 bp, Artificial sequence
  Single strand DNA oligonucleotide
gtccacgtcagcaatcatcct

SEQ ID NO: 17 - DNA, 20 bp, Artificial sequence
  Single strand DNA oligonucleotide
tctagtgatccctgagaagt

SEQ ID NO: 18 - DNA, 20 bp, Artificial sequence
  Single strand DNA oligonucleotide
acgcccttaatggcagtgat

SEQ ID NO: 19 - DNA, 21 bp, Artificial sequence
  Single strand DNA oligonucleotide
gtatgactccactcacggcaa

SEQ ID NO: 20 - DNA, 20 bp, Artificial sequence
  Single strand DNA oligonucleotide
ccattctcggccttgactgt

SEQ ID NO: 21 - DNA, 22 bp, Artificial sequence
  Single strand DNA oligonucleotide
tgaggagaagcttgggatgaaa

SEQ ID NO: 22 - DNA, 19 bp, Artificial sequence
  Single strand DNA oligonucleotide
gccctgagtctcctgaagc

SEQ ID NO: 23 - DNA, 22 bp, Artificial sequence
  Single strand DNA oligonucleotide
tgaggagaagcttgggatgaaa

SEQ ID NO: 24 - DNA, 19 bp, Artificial sequence
  Single strand DNA oligonucleotide
gccctgagtctcctgaagc

SEQ ID NO: 25 - DNA, 20 bp, Artificial sequence
  Single strand DNA oligonucleotide
tcaagcaggtggccagatct

SEQ ID NO: 26 - DNA, 22 bp, Artificial sequence
  Single strand DNA oligonucleotide
ggttatggctgatctcccaatc

SEQ ID NO: 27 - DNA, 20 bp, Artificial sequence
  Single strand DNA oligonucleotide
tgctgagtccgcagcaggtg

SEQ ID NO: 28 - DNA, 20 bp, Artificial sequence
  Single strand DNA oligonucleotide
gctggcaggctctggggaag

SEQ ID NO: 29 - DNA, 20 bp, Artificial sequence
  Single strand DNA oligonucleotide
ggagttaagacagcgcttgg

SEQ ID NO: 30 - DNA, 20 bp, Artificial sequence
  Single strand DNA oligonucleotide
actgggtccaagttgtccag

SEQ ID NO: 31 - DNA, 20 bp, Artificial sequence
  Single strand DNA oligonucleotide
ctgcaagaggtcctgtcttc

SEQ ID NO: 32 - DNA, 20 bp, Artificial sequence
  Single strand DNA oligonucleotide
caatcagagctcggcgagtc

SEQ ID NO: 33 - DNA, 20 bp, Artificial sequence
  Single strand DNA oligonucleotide
gcctttattgcttccagcag

SEQ ID NO: 34 - DNA, 20 bp, Artificial sequence
  Single strand DNA oligonucleotide
tgagacagcaaaaccgtctg

SEQ ID NO: 35 - DNA, 20 bp, Artificial sequence
  Single strand DNA oligonucleotide
gtagctcccagagaggggaa

SEQ ID NO: 36 - DNA, 20 bp, Artificial sequence
  Single strand DNA oligonucleotide
ctctagagggccagggactt

SEQ ID NO: 37 - DNA, 18 bp, Artificial sequence
  Single strand DNA oligonucleotide
gatgcgtccaccaagaag

SEQ ID NO: 38 - DNA, 18 bp, Artificial sequence
  Single strand DNA oligonucleotide
agttgaagttgccgtcag

SEQ ID NO: 39 - DNA, 20 bp, Artificial sequence
  Single strand DNA oligonucleotide
gaactgggggaggattgtgg

SEQ ID NO: 40 - DNA, 20 bp, Artificial sequence
  Single strand DNA oligonucleotide
acttcacttgtggcccagat

SEQ ID NO: 41 - DNA, 19 bp, Artificial sequence
  Single strand DNA oligonucleotide
ctctggtttcggtgggtgt

SEQ ID NO: 42 - DNA, 21 bp, Artificial sequence
  Single strand DNA oligonucleotide
tccagagtccattgattcgct

SEQ ID NO: 43 - DNA, 20 bp, Artificial sequence
  Single strand DNA oligonucleotide
ccatggggaaggtgaaggtc

SEQ ID NO: 44 - DNA, 20 bp, Artificial sequence
  Single strand DNA oligonucleotide
agtgatggcatggactgtgg

SEQ ID NO: 45 - DNA, 21 bp, Artificial sequence
  Single strand RNA oligonucleotide
gcaguucaacaaggcacaatt

SEQ ID NO: 46 - DNA, 21 bp, Artificial sequence
  Single strand RNA oligonucleotide
uugugccuuguugaacugctt

SEQ ID NO: 47 - DNA, 21 bp, Artificial sequence
  Single strand DNA oligonucleotide
catggctgaagtgaagcttgg

SEQ ID NO: 48 - DNA, 19 bp, Artificial sequence
  Single strand DNA oligonucleotide
tctcggtttttcctgtttg

SEQ ID NO: 49 - DNA, 63 bp, Artificial sequence
  Single strand DNA oligonucleotide
atcccgcagattctgcctctgttgttcaagagacaacagaggcagaatctgcttttttggaaa

SEQ ID NO: 50 - DNA, 63 bp, Artificial sequence
  Single strand DNA oligonucleotide
gcttttccaaaaaagcagattctgcctctgttgtctcttgaacaacagaggcagaatctgcgg